(19) United States
(12) Patent Application Publication
(10) Pub. No.: US 2026/0218283 A1
(43) Pub. Date: Jul. 30, 2026

(54) NUCLEIC ACID QUANTIFICATION METHOD AND REAGENT FOR QUANTIFYING NUCLEIC ACID

(71) Applicant: HITACHI HIGH-TECH CORPORATION, Tokyo (JP)

(72) Inventor: Noriyuki SUMIDA, Tokyo (JP)

(21) Appl. No.: 19/142,655

(22) PCT Filed: Jan. 19, 2023
(86) PCT No.: PCT/JP2023/001540

§ 371 Date: Jun. 24, 2025

(51) Int. Cl. C12Q 1/6851; C12Q 1/6827; C12Q 1/6853; C12Q 1/6876; G01N 27/447

(52) U.S. Cl. CPC C12Q 1/6851; C12Q 1/6827; C12Q 1/6853; C12Q 1/6876; G01N 27/44726; C12Q 2600/156

(57) ABSTRACT

To provide a nucleic acid quantification method and a reagent for quantifying nucleic acid that can quantify a proportion of a mutant-type base sequence to a wild-type base sequence contained in a sample with high accuracy, even when the mutant-type base sequence contained in the sample are present in extremely small amounts. A nucleic acid quantification method includes a step of amplifying polynucleotides using a wild-type primer set and a mutant-type primer set, and a step of fractionating the polynucleotides by electrophoresis, and a step of determining a proportion of the mutant-type polynucleotides to the wild-type polynucleotides. The wild-type forward primer has a base sequence complementary to a wild-type target base sequence at a 3′-terminal side, and the mutant-type forward primer has a base sequence complementary to a mutant target base sequence at a 3′-terminal side. The plurality of wild-type forward primers have mutually different molecular structures that result in different mobilities in the electrophoresis. The reagent for quantifying nucleic acid includes a plurality of wild-type forward primers, a mutant-type forward primer, and a reverse primer.

The present invention relates to a nucleic acid quantification method for quantifying a proportion of a mutant-type base sequence to a wild-type base sequence contained in a sample, and a reagent for quantifying nucleic acid.

### BACKGROUND ART

Malignant tumors are known to be caused by genetic mutations. In the early stages of cancer, the mutations in genes related to a cancer are found in a small portion of the cells that make up a tissue. Early stage mutations are mutations that occur at an extremely low frequency and are often point mutations. The accumulation of such low frequency mutations is thought to be related to cancer progression and risk.

Information on the occurrence status of low frequency mutations is an important indicator for determining the type and dosage of anticancer drugs used for treatment. Data quantifying the low frequency mutations is expected to contribute to the follow-up of patient prognosis, disease prediction, development of treatment methods, and streamlining of medical administration. The low frequency mutations occur in only a very small number of abnormal cells among many normal cells of the wild type. Therefore, it is necessary to eliminate the effects of sampling errors, measurement errors, replication errors, and the like, and perform accurate quantification.

A quantitative method for the low frequency mutations is to apply DNA sequencing technology to DNA or cDNA libraries created from samples. The wild-type base sequence and the mutant-type base sequence amplified by PCR are quantified using fluorescent labeling, radioactive labeling, or the like. The occurrence status of low frequency mutations is calculated as a proportion of a mutant-type base sequence to a wild-type base sequence.

Patent document 1 describes a method that includes testing for presence of changes in a proline-rich membrane-spanning protein 2 (PRRT2) gene. Assays for detecting changes in the PRRT2 gene include DNA sequencing, DNA hybridization, and electrophoretic assays. Patent document 2 describes a method for determining presence or absence of mutations in the human CD36 gene in individuals (see claim 40 and the like). In this method, a microarray screening is performed to hybridize a predetermined probe with a target nucleic acid. Intensity of signals generated from hybrid formation is measured by autoradiography, fluorescence analysis, or the like, and signals from mutant and normal cDNA are quantitatively compared.

### CITATION LIST

### Nonpatent Literature

Patent Literature 1: Japanese Unexamined Patent Application Publication (Translation of PCT Application) No. 2014-533939
Patent Literature 2: Japanese Unexamined Patent Application Publication (Translation of PCT Application) No. 2003-529315

### SUMMARY OF INVENTION

### Technical Problem

In order to evaluate occurrence status of the low frequency mutations in the base sequence, it is necessary to accurately quantify the proportion of the mutant-type base sequence to the wild-type base sequence. The quantitative method requires an ability to simultaneously quantify the wild-type base sequence, which is relatively abundant in the sample, and the mutant-type base sequence, which is relatively scarce. This is because different measurement systems are affected by sampling errors, measurement errors, and other factors. For example, when there is a 10% error, the low frequency mutations with an occurrence rate of 10% or less cannot be identified. In addition, the detection sensitivity for relatively small amounts of trace components must be ensured.

However, conventional nucleic acid quantification methods, including those using DNA sequencing technology, have the problem that the detection range of the detection target is limited by the performance of the detector. When analyzing quantitative targets labeled with fluorescent labeling, radioactive labeling, or the like, there is not only a detection sensitivity for trace components as the lower limit, but also a detection limit for major ingredients as an upper limit. When attempting to ensure the detection sensitivity for trace components, the detection signal for major ingredients may exceed the detection range of the detector.

When the mutant-type base sequence contained in the sample is in high concentration, the mutant-type base sequence can be detected with high accuracy. However, when the wild-type base sequence is also present in high concentrations, the detection signal derived from the wild-type base sequence will overshoot the detection range and cannot be accurately quantified. On the other hand, when the sample is diluted, the relatively small number of mutant-type base sequences cannot be quantified with high sensitivity. The insufficient detection sensitivity makes it susceptible to replication errors, making it impossible to accurately evaluate the low-frequency mutations present only in extremely small amounts among the large amount of wild-type base sequences.

In Patent Document 1, in an electrophoretic assay, primers for multiple exons are amplified simultaneously and evaluated simultaneously on a single electrophoresis gel. The amplification fragments across each exon are designed to be of different sizes. However, Patent Documents 1 and 2 do not specifically disclose a method for quantitatively determining a proportion of extremely small amount of a base sequence, such as a low frequency mutation.

Therefore, the present invention aims to provide a nucleic acid quantification method and a reagent for quantifying nucleic acid that can quantify a proportion of a mutant-type base sequence to a wild-type base sequence contained in a sample with high accuracy, even when the mutant-type base sequence contained in the sample are present in extremely small amounts.

### Solution to Problem

In order to solve the above problems, one aspect of a nucleic acid quantification method according to the present invention is a nucleic acid quantification method for quantifying a proportion of a mutant-type base sequence to a wild-type base sequence contained in a sample, and includes: a step of amplifying wild-type polynucleotides containing wild-type target base sequences and mutant-type polynucleotides containing mutant-type target base sequences by PCR using a wild-type primer set including a plurality of wild-type forward primers complementary to the wild-type target base sequence and a reverse primer complementary to a complementary strand of the target base sequence, and a mutant-type primer set including a mutant-type forward primer complementary to a mutant target base sequence and a reverse primer complementary to a complementary strand of the target base sequence; and a step of fractionating the wild-type polynucleotides and the mutant-type polynucleotides by electrophoresis to determine the proportion of the mutant-type polynucleotides to the wild-type polynucleotides. The wild-type forward primer has a base sequence complementary to the wild-type target base sequence at a 3′-terminal side, the mutant-type forward primer has a base sequence complementary to the mutant-type target base sequence at the 3′-terminal side, and the plurality of wild-type forward primers have mutually different molecular structures that result in different mobilities in the electrophoresis.

Further, one aspect of the nucleic acid quantification method according to the present invention is a nucleic acid quantification method for quantifying a proportion of a mutant-type base sequence to a wild-type base sequence contained in a sample, and includes: a step of hybridizing, to a wild-type polynucleotide containing the wild-type target base sequence and a mutant-type polynucleotide containing the mutant-type target base sequence, a wild-type probe set including a plurality of wild-type probes complementary to a wild-type target base sequence and a fragment complementary to an adjacent base sequence adjacent to a 5′-terminal side of the target base sequence and a mutant-type probe set including a mutant-type probe complementary to a mutant-type target base sequence and a fragment complementary to an adjacent base sequence adjacent to a 5′-terminal side of the target base sequence; a step of producing a wild-type polynucleotide including the wild-type target base sequence, by ligating the wild-type probe and the fragment hybridized to the wild-type polynucleotide and producing a wild-type polynucleotide including the wild-type target base sequence by ligating the mutant-type probe and the fragment hybridized to the mutant-type polynucleotide; and a step of fractionating the wild-type polynucleotide and the mutant-type polynucleotide by electrophoresis to determine a proportion of the mutant-type polynucleotide to the wild-type polynucleotide. The wild-type probe has a base sequence complementary to the wild-type target base sequence at a 3′-terminal side, the mutant-type probe has a base sequence complementary to the mutant-type target base sequence at a 3′-terminal side, and the plurality of wild-type probes have mutually different molecular structures that result in different mobilities in the electrophoresis.

Further, one aspect of a reagent for quantifying nucleic acid according to the present invention is a reagent for quantifying nucleic acid that quantifies a proportion of a mutant-type base sequence to a wild-type base sequence contained in a sample, and includes: a plurality of wild-type forward primers complementary to a wild-type target base sequence; a mutant-type forward primer complementary to a mutant-type target base sequence; and a reverse primer complementary to a complementary strand of the target base sequence. The wild-type forward primer has a base sequence complementary to the wild-type target base sequence at a 3′-terminal side, and the mutant-type forward primer has a base sequence complementary to the mutant target base sequence at a 3′-terminal side, and the plurality of wild-type forward primers have mutually different molecular structures that result in different mobilities in the electrophoresis.

Further, one aspect of a reagent for quantifying nucleic acid according to the present invention is a reagent for quantifying nucleic acid for quantifying nucleic acid that quantifies a proportion of a mutant-type base sequence to a wild-type base sequence contained in a sample, and includes: a plurality of wild-type probes complementary to a wild-type target base sequence; a plurality of mutant-type probes complementary to a mutant-type target base sequence; and a fragment complementary to an adjacent base sequence adjacent to a 5′-terminal side of the target base sequence. The wild-type probe has a base sequence complementary to the wild-type target base sequence at a 3′-terminal side, the mutant-type probe has a base sequence complementary to the mutant-type target base sequence at a 3′-terminal side, and the plurality of wild-type probes have mutually different molecular structures that result in different mobilities in the electrophoresis.

### Advantageous Effects of Invention

According to the present invention, it is possible to provide a nucleic acid quantification method and a reagent for quantifying nucleic acid that can quantify a proportion of a mutant-type base sequence to a wild-type base sequence contained in a sample with high accuracy, even when the mutant-type base sequence contained in the sample are present in extremely small amounts.

### BRIEF DESCRIPTION OF DRAWINGS

FIG. 1 is a flow diagram showing a nucleic acid quantification method according to a first embodiment of the present invention.

FIG. 2 is a schematic diagram showing a wild-type detection system that detects a wild-type target base sequence and a mutant-type detection system that detects a mutant-type target base sequence.

FIG. 3 is diagram showing an example of a result of a quantitative analysis of fractions fractionated by electrophoresis.

FIG. 4 is a schematic diagram showing a wild-type detection system for detecting a wild-type target base sequence and a mutant-type detection system for detecting a mutant-type target base sequence used in a test area.

FIG. 5 is a schematic diagram showing a wild-type detection system for detecting a wild-type target base sequence and a mutant detection system for detecting a mutant-type target base sequence used in a control area.

FIG. 6 shows an example of a result of a quantitative analysis in the control area.

FIG. 7 shows an example of a result of a quantitative analysis in the test area.

FIG. 8 is a flow diagram showing a nucleic acid quantification method according to a second embodiment of the present invention.

FIG. 9 is a schematic diagram showing a wild-type detection system for detecting a wild-type target base sequence and a mutant-type detection system for detecting a mutant-type target base sequence.

FIG. 10 shows an example of a result of a quantitative analysis of fractions fractionated by electrophoresis.

### DESCRIPTION OF EMBODIMENTS

The nucleic acid quantification method and the reagent for quantifying nucleic acid according to an embodiment of the present invention will be described below with reference to the drawings. In the following figures, the same symbols are used for common components, and duplicate explanations are omitted.

### First Embodiment: Nucleic Acid Quantification Method

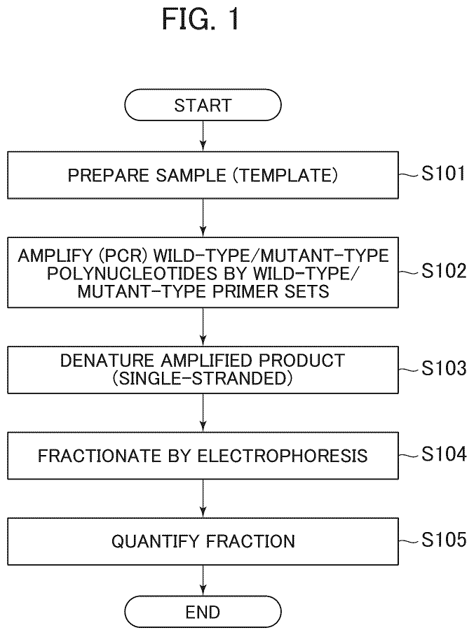

FIG. 1 is a flow diagram showing a nucleic acid quantification method according to the first embodiment of the present invention.

As shown in FIG. 1, the nucleic acid quantification method according to the first embodiment includes Step S101 of preparing a sample containing a template, Step S102 of amplifying polynucleotides, Step S103 of denaturing the amplified product, Step S104 of fractionating the denatured product by electrophoresis, and Step S105 of quantitatively analyzing the fractionated fractions.

The nucleic acid quantification method according to the first embodiment relates to a method for quantifying a proportion of a mutant-type base sequence to a wild-type base sequence contained in a sample. Examples of sample include a mixture of polynucleotides containing a wild-type base sequence and polynucleotides containing a mutant-type base sequence, such as a nucleic acid solution containing nucleic acids derived from mutually different cells. In this nucleic acid quantification method, the proportion of the mutant type to the wild type is determined for a predetermined target base sequence contained in a sample.

In this specification, the wild-type base sequence means the normal base sequence that is relatively abundant in relation to genes, intergenic regions, and the like. The wild-type base sequence is not limited to the base sequence present naturally, but may also be an artificially designed base sequence. A mutant-type base sequence refers to an abnormal base sequence that is relatively rare and has one or more bases that have been substituted (point mutation), deleted, or inserted relative to the wild-type base sequence, which is relatively abundant.

The target base sequence for analysis may be a base sequence on a gene, a base sequence in an intergenic region, or an artificially designed base sequence. The length of the target base sequence is not particularly limited as long as it can be recognized by complementary nucleic acid fragments. The mutant-type base sequence may have a single base mutation, multiple consecutive base mutations, and/or multiple intermittent base mutations.

In the nucleic acid quantification method according to the first embodiment, the wild-type base sequence and the mutant-type base sequence contained in the sample are each detected by a predetermined primer complementary to the base sequence. Then, PCR is performed using the primer set, and the amplified PCR products are fractionated by electrophoresis to quantify each fraction.

The primer set includes a forward primer complementary to the target base sequence and a 3′-terminal side of the sequence, and a reverse primer complementary to a 3′-terminal side of the complementary base sequence complementary to the target base sequence. The target base sequence of the analytical target is detected by the forward primer that selectively binds to the base sequence.

In the nucleic acid quantification method according to the first embodiment, a plurality of primers having a base sequence complementary to the wild-type target base sequence and having mutually different mobilities in electrophoresis are used as a wild-type forward primer that recognizes the wild-type base sequence. In addition, as a mutant-type forward primer that recognizes the mutant-type base sequence, a primer having a base sequence complementary to the mutant-type target base sequence and having a mobility different from that of the wild type in the electrophoresis is used.

The PCR products are fractionated by the electrophoresis, and each fraction is quantified to obtain quantitative results of the PCR products using the wild-type forward primer and the reverse primer and quantitative results of the PCR products using the mutant-type forward primer and the reverse primer. Based on these quantitative results, the proportion of the mutant-type base sequence to the wild-type the base sequence can be determined.

In general, the low frequency mutation found in the early stages of cancer are difficult to quantify accurately because only a very small number of abnormal cells are produced among many normal cells of the wild type. In order to evaluate the occurrence status of low frequency mutations, it is necessary to accurately quantify the proportion of the mutant-type base sequences to the wild-type base sequences. High detection sensitivity is required because nucleic acids with low frequency mutations are present only in the extremely small amounts. In addition, from the perspective of eliminating the effects of sampling errors, measurement errors, and the like, it is desirable to quantitatively analyze the relatively abundant wild-type base sequence and the relatively scarce mutant-type base sequence collectively at the same time.

However, in the conventional nucleic acid quantification methods, the detection range of the detection target is limited by the performance of the detector. A common nucleic acid quantification method is based on a DNA sequencing technology, in which a nucleic acid labeled with a fluorescent labeling agent is subjected to spectroscopic analysis. In the quantitative methods that detect and quantify such labels, there is not only a detection sensitivity for a trace component as the lower limit, but also a detection limit for a major ingredient as the upper limit. When the detection target is contained in extremely large quantities, the signal intensity, such as fluorescence intensity, will exceed the detection limit of the detector, making accurate quantification impossible.

Therefore, it is difficult for the conventional nucleic acid quantification methods to collectively quantify simultaneously the relatively large number of wild-type base sequences and the relatively small number of mutant-type base sequences in the sample. When attempting to ensure the detection sensitivity for trace components, the detection signal of the major ingredient overshoots the detection limit and does not fall within the detection range by the detector. On the other hand, when the sample is diluted to accurately quantify the major ingredient, the detection sensitivity for the trace component cannot be obtained.

In contrast, when the plurality of primers with mutually different mobilities in the electrophoresis are used as the wild-type forward primers, the PCR using the polynucleotides containing the wild-type target base sequence as a template can produce a plurality of types of polynucleotides derived from the wild-type base sequences with different mobilities in the electrophoresis. Since the plurality of types of polynucleotides can be fractionated into a plurality of fractions by the electrophoresis, the detection signals derived from the relative abundance of the wild-type base sequences can be split into smaller detection signals for respective fractions.

Therefore, even when the quantitative detection range of the detection target is limited by the performance of the detector, it is possible to simultaneously and collectively quantify the relatively large number of wild-type base sequences and the relatively small number of mutant-type base sequences in the sample. Even when evaluating the low frequency mutations that occur at extremely low frequencies, the detection sensitivity for relatively few mutant-type base sequences can be maintained, while the detection signals derived from relatively many wild-type base sequences can be easily brought within the detection range of the detector. Therefore, the proportion of the mutant-type base sequences to the wild-type base sequences in the sample can be quantified with high accuracy.

### (Step S101)

Step S101 is a step of preparing a sample of the analytical target for which the proportion of the mutant-type base sequence to the wild-type base sequence is to be quantified, and which contains polynucleotides containing the target base sequence that will serve as the template for PCR.

The analytical target sample can be a nucleic acid solution in which polynucleotides containing a predetermined target base sequence are dissolved. It is sufficient that the template polynucleotide contains at least one of the wild-type target base sequence and the mutant-type target base sequence. The wild-type target base sequence and the mutant-type target base sequence may be on different molecules or on the same molecule as one another. For example, when the target base sequence constitutes a repeating sequence, the proportion of the mutant-type base sequences on the same molecule as one another can be determined.

In addition to the polynucleotides containing the target base sequence, it is preferred that the analytical target sample contain a buffering agent that exhibits a pH buffering effect and/or a chelating agent such as EDTA. The analytical target sample is preferred to be free of active nucleases. The analytical target sample is preferred to be adjusted to be pH 7.5 or more and pH 8.5 or less.

The polynucleotides containing the target base sequence is preferred to be purified against other cellular components such as proteins, lipids, and salts. The polynucleotides can be purified using common purification methods such as alkaline extraction, phenol-chloroform extraction, and density gradient centrifugation, as well as commercially available purification kits that include purification columns.

The polynucleotides containing the target base sequence may be extracted from a specimen or artificially prepared. For example, the polynucleotides containing the target base sequence can be prepared as genomic DNA libraries, cDNA libraries, and the like by extracting and purifying DNA, RNA, or fragments thereof from tissue fragments, cell groups, and the like collected from the specimen. It can also be prepared as a DNA library, and the like through an artificial processing and reactions.

For example, it is possible to prepare a template selected for any purpose by artificial processes and reactions. DNA libraries constructed for higher level mutations such as chromosomal aberrations and exon aberrations can be used for the analytical target for the lower level mutation such as base substitutions, deletions, and insertions. Such DNA libraries include a ligation product from a Multiplex Ligation-dependent Probe Amplification (MLPA) method.

### (Step S102)

Step S102 is a step of amplifying the polynucleotides containing the target base sequence by the polymerase chain reaction (PCR) using the template and the primer set.

In Step S102, the wild-type polynucleotides containing the wild-type target base sequence and the mutant-type polynucleotides containing the mutant-type target base sequence are amplified by the PCR using the wild-type primer set and the mutant-type primer set. In Step S102, the wild-type target base sequence is detected by the forward primer that constitutes the wild-type primer set. The mutant-type target base sequence is also detected by the forward primer that constitutes the mutant-type primer set.

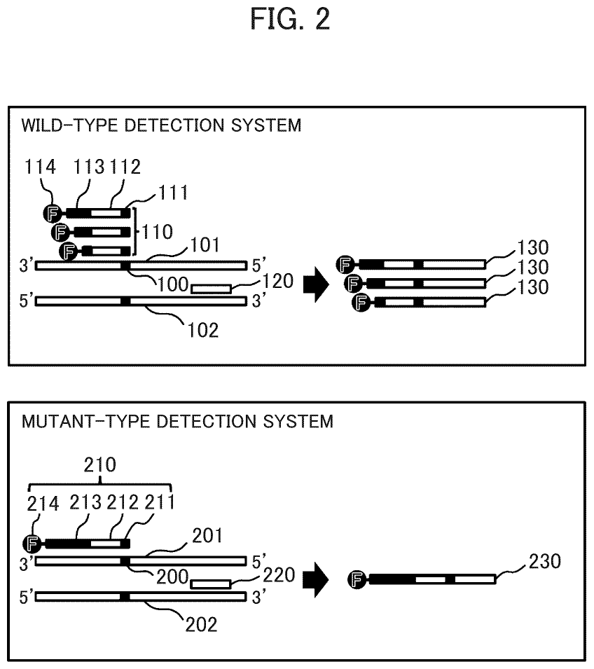

FIG. 2 is a schematic diagram showing the wild-type detection system that detects the wild-type target base sequence and the mutant-type detection system that detects the mutant-type target base sequence. The upper side of FIG. 2 shows the template containing the wild-type target base sequence, the wild-type primer set, and the PCR products amplified by them. The lower part of FIG. 2 shows the template containing the mutant-type target base sequence, the mutant-type primer set, and the PCR products amplified by them.

As shown in the upper part of FIG. 2, a wild-type polynucleotide (a target strand) 101, which contains a wild-type target base sequence 100 in the sample, is the template for the PCR in the wild-type detection system. The wild-type detection system, which detects the wild-type target base sequence, is composed of wild-type primer sets 110, 120. The wild-type primer sets 110, 120 are composed of the plurality of wild-type forward primers (F primers) 110 and a reverse primer (R primers) 120.

The wild-type F primer 110 binds to the target strand 101 containing the wild-type target base sequence 100. The wild-type F primer 110 has a complementary base sequence to the target strand 101. The wild-type F primer 110 is composed of a plurality of types designed to differ from one another in the mobility in the electrophoresis. The plurality of wild-type F primers 110 each selectively bind to a region containing the wild-type target base sequence 100 of each target strand 101 in the sample.

The R primer 120 binds to a complementary strand 102, which is complementary to the target strand 101 containing the wild-type target base sequence 100. The R primer 120 has a sequence complementary to the complementary strand 102. The R primer 120 binds selectively to the 3′-terminal side of the complementary base sequence of the complementary strand 102 that is complementary to the target base sequence 100 in order to amplify a certain length.

As shown in the lower part of FIG. 2, a mutant-type polynucleotide (a target strand) 201 containing a mutant-type target base sequence 200 in the sample is the template for the PCR in the mutant-type detection system. The mutant-type detection system for detecting the mutant-type base sequence is composed of mutant-type primer sets 210, 220. The mutant-type primer sets 210, 220 are composed of a mutant-type forward primer (F primer) 210 and a reverse primer (R primer) 220.

The mutant-type F primer 210 binds to the target strand 201 containing the mutant-type target base sequence 200. The mutant-type F primer 210 has a complementary base sequence to the target strand 201. The mutant-type F primer 210 is composed of at least one type designed to have a different mobility in the electrophoresis from the wild-type F primer 110. The mutant-type F primer 210 selectively binds to a region containing the mutant-type target base sequence 200 of each target strand 201 contained in the sample.

The R primer 220 binds to a complementary strand 202 complementary to the target strand 201 including a mutant-type target base sequence 200. The R primer 220 has a sequence complementary to the complementary strand 202. In order to amplify a certain length, the R primer 220 selectively binds to the 3′-terminal side of the complementary strand 202 with respect to the complementary base sequence complementary to the target base sequence 200.

In Step S102, the wild-type detection system and the mutant-type detection system are reacted in the same reaction system. To the same reaction solution for the PCR, the plurality of wild-type F primers 110, the mutant-type F primer 210, and at least one type of R primers 120, 220, which mutually differ in the mobility in the electrophoresis are added in substantially equal amounts to one another. At least one type of R primers 120, 220 can be added for both the wild-type and the mutant-type.

In Step S102, the wild-type primer sets 110, 120 are used to amplify a wild-type polynucleotide 130 containing the wild-type target base sequence 100. As for the wild-type polynucleotides 130, a plurality of types with the mutually different mobilities in the electrophoresis are generated. A mutant-type polynucleotide 230 containing the mutant-type target base sequence 200 is amplified by the mutant-type primer sets 210, 220.

The PCR can be performed by adding a template polynucleotide, the wild-type primer sets 110, 120, the mutant-type primer sets 210, 220, the DNA polymerase with heat resistance, and the dNTP mixture to the reaction buffer solution according to the usual method. As a reaction buffer solution, an aqueous solution in which a buffering agent that shows a pH buffering effect, magnesium ions as a cofactor, and additives to be added as needed can be used.

Appropriate types of the DNA polymerase, such as TaqDNA polymerase, PfuDNA polymerase, TopDNA polymerase, or the like can be used. The dNTP mixture is a mixture of deoxynucleoside triphosphates and includes deoxyadenosine triphosphate (dATP), deoxythymidine triphosphate (dTTP), deoxyguanosine triphosphate (dGTP), and deoxycytidine triphosphate (dCTP).

Examples of the buffering agents include Tris-HCl Buffer, Tris-Acetate Buffer, HEPES Buffer, and phosphate buffers, such as disodium hydrogen phosphate, sodium dihydrogen phosphate, dipotassium hydrogen phosphate, and potassium dihydrogen phosphate. Examples of the additives include reducing agents such as 2-mercaptoethanol and dithiothreitol, surfactants such as TritonX-100 and Tween 20, polyethylene glycol, glycerol, BSA, gelatin betaine, formamide, dimethyl sulfoxide.

As for the PCR, a common thermal cycle reaction can be used. A typical thermal cycle includes a denaturation step of denaturing the polynucleotide to single stranded, an annealing step of annealing the primer to the polynucleotide, and an extension step of elongating the polynucleotide. The repetition of these steps synthesizes large amounts of polynucleotides suitable for quantitation.

The denaturation step is preferred to be, for example, at 96° C. for 30 seconds. The annealing step is preferred to be, for example, 50 to 60° C. for 30 seconds. The extension step is preferred to be, for example, 72° C. for 30 seconds. The denaturation step at the beginning of the cycle is preferred to be, for example, 1 to 10 min at 96° C. to dissociate the template into the single strands. The number of cycles for each step is preferred to be, for example, 25 cycles or more and 40 cycles or less.

The reaction conditions for the PCR can be appropriately adjusted according to the base lengths of the template and the primer, the GC content, and the performance of the detector used for quantification. The reaction conditions for the PCR include the concentrations of the template, the wild-type primer sets 110, 120, the mutant-type primer sets 210, 220, the DNA polymerase, and the additives, as well as the temperature and the time of each step and the number of cycles to repeat each step.

As shown in FIG. 2, the wild-type F primer 110 has a mutation recognition site 111, a target recognition site 112, a mobility correction site 113, and a labeled site 114. The mutation recognition site 111, the target recognition site 112, the mobility correction site 113, and the labeled site 114 are arranged in a state of being connected in this order from the 3′-terminal side of the wild-type F primer 110.

The mutant-type F primer 210 has a mutation recognition site 211, a target recognition site 212, a mobility correction site 213, and a labeled site 214. The mutation recognition site 211, the target recognition site 212, the mobility correction site 213, and the labeled site 214 are arranged in a state of being connected in this order from the 3′-terminal side of the mutant-type F primer 210.

The mutation recognition sites 111, 211 are sites for recognizing the target base sequences 100, 200 and identifying whether the target base sequences 100, 200 are mutated or not. The mutation recognition sites 111, 211 can be formed with polynucleotides of any degree of polymerization.

The mutation recognition site 111 of the wild-type F primer 110 is a complementary base sequence to the wild-type target base sequence 100. The mutation recognition site 111 of the wild-type F primer 110 is provided in a common base sequence among the plurality of wild-type F primers 110.

The mutation recognition site 211 of the mutant-type F primer 210 is a complementary base sequence to the mutant-type target base sequence 200. The mutation recognition site 211 of the mutant-type F primer 210 is preferred to be formed of the same base sequence as the mutation recognition site 111 of the wild-type F primer 110, except for the loci complementary to the loci that have been mutated. Such a base sequence facilitates the adjustment of the mobility in the electrophoresis.

According to the mutation recognition sites 111, 211, it is identified whether the target base sequence of the polynucleotide contained in the sample is the wild-type base sequence or the mutant-type base sequence. When the mutation recognition sites 111 of the wild-type F primer 110 is easy to bind and the PCR product amplified by the wild-type primer sets 110, 120 is relatively large, the target base sequence in question is the wild-type. On the other hand, when the mutation recognition site 211 of the mutant-type F primer 210 is easy to bind and the PCR product amplified by the mutant-type primer sets 210, 220 is relatively large, the target base sequence in question is the mutant-type.

The lengths of the mutation recognition sites 111, 211 are not particularly limited, but 1 nt or more and 6 nt or less is preferred, and 1 nt or more and 5 nt or less is more preferred. Such a length increases the efficiency of selective annealing and thus reduces misidentification of the wild-type and the mutant-type.

The mutation recognition sites 111, 211 are provided at the 3′-terminal side of each of the F primers 110, 210. The nucleotide at the 3′-terminal side of each of the F primers 110, 210 is preferred to be in an arrangement that forms a hydrogen bonding with the base that produces the polymorphism due to the mutation in the target base sequence 100, 200. This arrangement makes it difficult for the polynucleotides to elongate when the mutation recognition sites 111, 211 undergo the mismatched annealing. Artifacts due to the mismatched annealing are reduced, allowing accurate identification of the wild-type and the mutant-type.

The target recognition sites 112, 212 are sites for recognizing a common sequence other than the target base sequences 100, 200 and identifying the position of the amplification in the PCR. The target recognition sites 112, 212 are connected to the 5′-terminal sides of the mutation recognition sites 111, 211. The target recognition sites 112, 212 can be formed with polynucleotides of any degree of polymerization.

The target recognition sites 112, 212 are complementary base sequences to the common sequence adjacent to the target base sequences 100, 200. The common sequence is the base sequence that is common to the target strand 101 with the wild-type target base sequence 100 and the target strand 201 with the mutant-type target base sequence 200.

The target recognition site 112 of the wild-type F primer 110 is provided in a common sequence among the plurality of wild-type F primers 110. The target recognition site 212 of the mutant-type F primer 210 is preferred to be formed with the same base sequence as the target recognition site 112 of the wild-type F primer 110. Such a base sequence facilitates the adjustment of the mobility in the electrophoresis.

According to the target recognition sites 112, 212, the target strands 101, 201 having the target base sequences 100, 200 contained in the sample and amplification start positions in PCR are identified. Since the position-selective bonding of each of the F primers 110, 210 to the template is ensured, the target base sequences 100, 200 can be properly identified by the mutation recognition sites 111, 211.

The lengths of the target recognition sites 112, 212 are not particularly limited, but 10 nt or more and 50 nt or less is preferred, and 10 nt or more and 30 nt or less is more preferred. Such a length increases the efficiency of the selective annealing to the target strands 101, 201, thus reducing misidentification of the template and misidentification of the amplification start position.

The mobility correction sites 113, 213 are sites for correcting the mobility in the electrophoresis for the quantitative target polynucleotides 130, 230, which are the PCR products. The mobility correction sites 113, 213 are connected to the 5′-terminal side of the target recognition sites 112, 212. The mobility correction sites 113, 213 can be formed with polynucleotides of any degree of polymerization or polymers that are non-polynucleotides of any degree of polymerization.

The mobility correction site 113 of the wild-type F primer 110 is provided in the molecular structures with the mutually different mobilities in the electrophoresis among the plurality of wild-type F primers 110. The mobility correction site 213 of the mutant-type F primer 210 is provided in the molecular structure with the mobility in the electrophoresis different from the mobility correction site 113 of the wild-type F primer 110.

According to the mobility correction sites 113, 213, the PCR using each of the primer sets 110, 120, 210, 220 can synthesize the plurality of types of polynucleotides 130, 230 with the mutually different mobilities in the electrophoresis. When the PCR products are fractionated into fractions by the electrophoresis and the quantification is performed by detecting the labeling of each fraction, the wild-type polynucleotide 130 containing a relatively large number of wild-type target base sequences 100 can be divided into the plurality of fractions. Since the detection signal derived from the wild-type is subdivided into respective fractions, the detection signal derived from the wild-type can be easily brought within the detection range by the detector. The difference in the mobility in the electrophoresis between wild-type and the mutant-type allows easily distinguishing between the relatively more abundant wild-type and the relatively less abundant mutant-type.

The mobility correction sites 113, 213 can be provided in the mutually different molecular lengths, molecular weights, molecular structures, and the like such that the mobilities in the electrophoresis between the plurality of wild-type F primers 110 or between the wild-type F primer 110 and the mutant-type F primer 210 are mutually different.

The mobility correction sites 113, 213 are preferably provided in a molecular structure with a common part between the plurality of wild-type F primers 110 or between the wild-type F primer 110 and the mutant-type F primer 210. The mobility in the electrophoresis is preferred to be adjusted by an additional part connected to the common part. The common part is preferred to be located on the side of the target recognition sites 112, 212 at the mobility correction sites 113, 213. Such a molecular structure allows for easy adjustment of the mobility in the electrophoresis while ensuring the selective binding to the target base sequences 100, 200.

The mobility correction sites 113, 213 may be formed solely of the polynucleotides, solely of the non-polynucleotide polymers, or a combination of the two. Examples of non-polynucleotides include polyamino acids; polyalkylene glycols such as polyethylene glycol and polypropylene glycol; polysaccharides that form sugar chains; and sugar nucleotides such as poly(ADP-ribose).

The mobility correction sites 113, 213 may be provided in a linear or branched molecular structure, but it is preferred that they be provided in a branched molecular structure with branches for at least some of the plurality of wild-type F primers 110. The branched shape allows for more reliable formation of differences in the mobility in the electrophoresis. This makes it possible to analyze the plurality of mutations in a single electrophoresis. In addition, the influence of charge and conformation during the electrophoresis is reduced, thus reducing the mobility errors.

As for the branched molecular structures, the branched polynucleotides can be formed, for example, by modifying the phosphate group of the nucleotide or the hydroxyl group of the ribose. The branched polyamino acids can also be formed by introducing lysine residue, aspartic acid residue, and glutamic acid residue as well as polyamines and polycarboxylic acids, into polyamino acids. The branched polyalkylene glycol can be formed by introducing various functional groups, such as polyfunctional amino groups, maleimide groups, and polyfunctional carboxyl groups, into a polyalkylene glycol.

The lengths of the mobility correction sites 113, 213 are not limited when formed by polynucleotides, but 10 nt or more and 100 nt or less is preferred. Such a length reduces the mismatched annealing of each of the F primers 110, 210 through the mobility correction sites 113, 213.

When formed with the polynucleotides, the mobility correction sites 113, 213 can be set to a suitable degree of polymerization difference of at least 1 nt or more between the plurality of wild-type F primers 110 or between the wild-type F primer 110 and the mutant-type F primer 210. A polymerization degree difference of 5 nt or more is preferred from the standpoint of separability in electrophoresis, and 10 nt or more is more preferred. For example, the wild-type F primer 110 can be composed of the plurality of types, such as 20 nt, 40 nt, 60 nt, or the like.

The length of the mobility correction site 213 of the mutant-type F primer 210 is preferred to be longer than the length of the mobility correction site 113 of the wild-type F primer 110. In general, the longer the molecular chain, the more likely the electrophoresis mobility is to vary and the more likely it is to produce noise in the detection signal. In contrast, when the mobility correction site 213 of the mutant-type F primer 210 is relatively long, the relatively short PCR product amplified by the wild-type F primer 110 will be less noisy. Accurate quantification can be performed because noise mixing due to the plurality of types of PCR products can be avoided.

The labeled sites 114, 214 are sites that label the polynucleotides to be quantified as the PCR products. The labeled sites 114, 214 can be formed with fluorescent dyes, radioisotopes, or the like. The labeled sites 114, 214 are preferred to be connected to the terminal side of the mobility correction sites 113, 213 opposite the target recognition sites 112, 212 when the fluorescent dyes are used.

According to the labeled sites 114, 214, the PCR using each of the primer sets 110, 120, 210, 220 can yield the wild-type polynucleotides 130 and the mutant-type polynucleotides 230 labeled with fluorescent dyes, radioactive isotopes, or the like. When the PCR products are fractionated into fractions by the electrophoresis, the label of each fraction can be detected and quantified. Any suitable type of fluorescent dye that can bind to the mobility correction sites 113, 213 and emit fluorescence of any wavelength can be used. With the fluorescent dyes, each of the F primers 110, 210 is easier to prepare and handle than when the radioisotopes are used.

Examples of fluorescent dyes include coumarin-based dyes such as aminomethylcoumarin, 7-hydroxy-4-methylcoumarin, 7-amino-4-methylcoumarin, and 7-acetoxy-4-methylcoumarin; fluorescein-based dyes such as 5-carboxyfluorescein, 6-carboxyfluorescein, 5-aminofluorescein, 6-aminofluorescein, fluorescein-5-isothiocyanate, fluorescein-6-isothiocyanate, and fluorescein-5-maleimide; rhodamine-based dyes such as rhodamine B, rhodamine 110, rhodamine 6G, 5-carboxyrhodamine 110, and 6-carboxyrhodamine 110; and other dyes such as nitrobenzoxadiazole, cyanine-based dyes, pyrene-based dyes, and dansyl-based dyes.

Any appropriate type of radioisotope can be used, such as radioisotopes of atoms constituting the atomic groups that bind to the mobility correction sites 113, 213, or radioisotopes of atoms constituting the wild-type F primer 110 and the mutant-type F primer 210. With the radioisotopes, inexpensive detectors can be used for detecting the labeling compared to the use of the fluorescent dyes.

Examples of radioactive isotopes that label the phosphate group of nucleotides include phosphorus 32 (³²P) and phosphorus 33 (³³p). The radioactive isotopes that label amino acids and other compounds connected to nucleotides include sulfur 35 (³⁵S), iodine 125 (¹²⁵I), and others. Examples of radioactive isotopes that label polyalkylene glycols and other compounds connected to nucleotides include tritium (³H), carbon-14 (¹⁴C), and other radioactive isotopes.

The labeled sites 114 of the wild-type F primer 110 may be labeled with the fluorescent dyes that emit fluorescence at mutually different wavelengths or at the same wavelength among the plurality of wild-type F primers 110.

When labeled with the fluorescent dyes that emit fluorescence at the different wavelengths, a wavelength-by-wavelength spectroscopic analysis can split the detection signal derived from the relatively large number of wild-type base sequences into smaller detection signals at different wavelengths. Since the ability to use a wider variety of primers makes it possible to collectively quantify the relatively more abundant wild-type base sequences and the relatively less abundant mutant-type base sequences contained in the sample simultaneously, the proportion of the mutant-type base sequences to the wild-type base sequences contained in the sample can be quantified with high accuracy.

The labeled site 114 of the wild-type F primer 110 may be labeled with a fluorescent dye that emits fluorescence at a wavelength different from the labeled site 214 of the mutant-type F primer 210, or they may be labeled with the fluorescent dyes that emit fluorescence at the same wavelength.

When labeled with the fluorescent dyes that emit fluorescence at the different wavelengths, a wavelength-by-wavelength spectroscopic analysis can accurately distinguish detection signals derived from the wild-type from those derived from the mutant-type. This enables the use of a wider variety of primers, and even when the mobilities in the electrophoresis of the labeled site 114 of the wild-type F primer 110 and the labeled site 214 of the mutant-type F primer 210 are similar to one another, the amount of the wild-type base sequence and the amount of the mutant-type base sequence in the sample can be accurately quantified.

The lengths of R primers 120, 220 are not limited, but 10 nt or more and 50 nt or less is preferred, and 10 nt or more and 30 nt or less is more preferred. Such a length increases the efficiency of the selective annealing to the complementary strands 102, 202, thus reducing misidentification of the template identification and misidentification of the end position of amplification.

### (Step S103)

Step S103 is a step of preparing the samples for the electrophoresis by denaturing the polynucleotides, which are amplified products amplified by the PCR, into single strands.

In Step S103, the wild-type polynucleotides 130 containing the wild-type target base sequence 100 amplified by the wild-type primer sets 110, 120, and the mutant-type polynucleotides 230 containing the mutant-type target base sequence 200 amplified by the mutant-type primer sets 210, 220 are dissociated into single strands suitable for the electrophoresis. The ions and other substances in the reaction solution are separated or diluted to prepare the sample suitable for electrophoresis.

The polynucleotides can be denatured by a common method such as chemical treatment, heat treatment, or a combination of these methods, or by a commercially available purification kit that includes a purification column. Examples of chemical treatments include the addition of a denaturant, the addition of a salt, and pH adjustment. Denaturants include, for example, formamide and urea.

In addition to the polynucleotides 130, 230 as the amplified product, the sample for the electrophoresis is preferred to contain a buffering agent that exhibits a pH buffering effect and a chelating agent such as EDTA. For example, the samples for the electrophoresis can be prepared by diluting a reaction buffer solution containing an amplified product with a denaturant-added buffer solution. The samples for the electrophoresis is preferred to be adjusted to pH 7.5 or more and pH 8.5 or less. Tris-acetate buffer and Tris-borate buffer are preferred buffering agents.

### (Step S104)

Step S104 is a step of fractionating the polynucleotides, which are amplified products amplified by the PCR, by electrophoresis.

In Step S104, the wild-type polynucleotides 130 containing the wild-type target base sequence 100 amplified by the wild-type primer sets 110, 120 and the mutant-type polynucleotides 230 containing the mutant-type target base sequence 200 amplified by the mutant-type primer sets 210, 220 are separated into fractions of each molecular weight by the electrophoresis.

The electrophoresis of the polynucleotides can be performed by capillary electrophoresis, gel electrophoresis, or the like. The capillary electrophoresis can be performed using capillary type electrophoresis equipment such as a sequencer equipped with an autosampler. The gel electrophoresis can be performed using an electrophoresis tank or similar apparatus with an agarose gel or a polyacrylamide gel being as a separation medium.

The electrophoresis of the polynucleotides is preferably performed by capillary electrophoresis from the viewpoint of the high resolution and quantitative and the collective loading and detection of the PCR products. From the viewpoint of utilizing the molecular sieving effect, it is more preferable to perform capillary gel electrophoresis using gel as the separation media. The capillary gel electrophoresis can be performed with gel-filled capillaries and polymer-dispersed samples.

As a capillary type electrophoresis device, a device provided with a separation part composed of capillaries, a power supply part that applies voltage to both ends of the capillary, and a detection part that detects the label of the sample fractionated by the capillary is preferred in that they allow the fractionation and the quantitative analysis to be performed continuously. The separation part can be a capillary made of a narrow tube formed of silica glass, borosilicate glass, or the like, coated with polyimide, or the like, with an unmodified inner surface or a capillary with a modified inner surface.

### (Step S105)

Step S105 is a step of performing a quantitative analysis of the fraction fractionated by the electrophoresis.

In Step S105, the wild-type polynucleotides 130 containing the wild-type target base sequence 100 and the mutant-type polynucleotides 230 containing the mutant-type target base sequence 200, which have been fractionated by molecular weight using the electrophoresis, are quantified for each fraction fractionated by the electrophoresis to determine the proportion of the mutant-type base sequence to the wild-type base sequence.

The proportion of the mutant-type base sequence to the wild-type base sequence can be derived by dividing the amount of the mutant-type polynucleotide by a sum of the amount of the wild-type polynucleotide and the mutant-type polynucleotide, based on the quantitative results of the wild-type polynucleotide and the mutant-type polynucleotide.

As the quantitative result, a peak height of the detection signal or a peak area of the detection signal may be used for quantification by detecting the label for each fraction, but from the perspective of the high accuracy quantification, the peak area of the detection signal is preferred. The proportion of the mutant-type base sequence to the wild-type base sequence can be derived by dividing the peak height or the peak area of the detection signal of the mutant-type polynucleotide by a sum of the peak heights or the peak areas of all the detected signals.

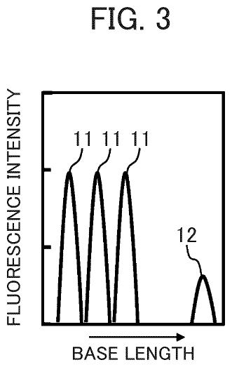

FIG. 3 shows an example of a quantitative result of the quantitative analysis of fractions fractionated by the electrophoresis. FIG. 3 shows the result of the spectroscopic analysis of the fluorescence of each fraction after PCR was performed using the fluorescent labeling primer sets and the amplified PCR products were fractionated by the electrophoresis. In FIG. 3, the horizontal axis indicates the base length of the PCR product. The vertical axis indicates the fluorescence intensity of the PCR product.

Reference signs 11 indicate the PCR products amplified by the wild-type primer sets 110, 120, resulting in the wild-type polynucleotide 130 containing a relatively large number of the wild-type target base sequences 100. Reference sign 12 indicates the result of the PCR product amplified by the mutant-type primer sets 210, 220, which is the mutant-type polynucleotide 230 containing the relatively few mutant-type target base sequences 200.

As shown in FIG. 3, the PCR products amplified by the wild-type primer sets 110, 120 produce the plurality of detection signals by using the plurality of wild-type F primers 110. Using the plurality of wild-type F primers 110 with the mutually different mobilities in the electrophoresis, the detection signal derived from the wild-type target base sequence 100 is split into the plurality of smaller signals. Therefore, the detection signal for each fraction can be easily within the lower limit of the detection range, which is above the detection sensitivity, and below the upper limit, which is the detection limit.

As shown in FIG. 3, the PCR products amplified by the mutant-type primer sets 210, 220 produce the different signal from the signal from the PCR product amplified by the wild-type primer sets 110, 120. The use of the mutant-type F primer 210 with the mobility in the electrophoresis different from the mobility of the wild-type F primer 110 allows the accurate quantification because the detection signal derived from the wild-type target base sequence 100 is distinguished from the detection signal derived from the mutant-type target base sequence 200.

Here, the quantitative method for quantifying the proportion of the mutant-type base sequences to the wild-type base sequences is described more specifically for genomic DNA.

The polynucleotide containing the wild-type target base sequence shall be the genomic DNA BRAF Wild Type Reference Standard (Horizon discovery) with the wild-type BRA F gene. The polynucleotides containing the mutant-type target base sequences include the genomic DNA with the mutant-type BRAF gene. BRAFV600K (manufactured by Horizon discovery) is to be used.

In the polynucleotides containing the wild-type target base sequences, the 140453136th base from the end of the short arm (p-arm) of chromosome 7 is adenine and the 140453137th base is cytosine. On the other hand, in the polynucleotides containing the mutant-type target base sequences, these loci are mutated to thymine.

In the quantitative analysis, first, the wild-type polynucleotides containing the wild-type target base sequences and the mutant-type polynucleotides containing the mutant-type target base sequences are amplified by the PCR using the wild-type primer sets and the mutant-type primer sets. The polynucleotide as the amplified product is then fractionated by the electrophoresis, and the fractionated fractions are subjected to the quantitative analysis to determine the proportion of the mutant-type base sequence to the wild-type base sequence.

The analytical target sample shall be a nucleic acid solution that simulates a low frequency mutation with a mutation rate of 10%. The nucleic acid solution was prepared by adding 9 ng of the wild-type polynucleotide and 1 ng of the mutant-type polynucleotide to the reaction buffer solution. The electrophoresis and the quantitative analysis shall be performed using the capillary electrophoresis equipment capable of quantitatively detecting the fluorescence from 10 to 100 RFU.

The quantitative analysis will evaluate the effectiveness of the test area compared with the control area. In the test area, the plurality of types of wild-type F primers designed to have the mutually different mobilities in the electrophoresis are used as the forward primers constituting the wild-type primer set. In the control area, one type of wild-type F primer is used as the forward primer constituting the wild-type primer set.

Table 1 shows specific examples of the primer sets for the wild-type detection system and the primer sets for the mutant-type detection system used in the test area.

TABLE 1

| | | | BASE LENGTH (nt) | | |
|---|---|---|---|---|---|
| | | | MOBILITY | TARGET | |
| PRIMER | | FLUORESCENT | CORRECTION | RECOGNITION | REACTION |
| SET | TYPE | DYE | SITE | SITE | PRODUCT |
| WILD | PRIMER F1 | FITC | 15 | 23 | 159 |
| TYPE | PRIMER F2 | FITC | 18 | 23 | 162 |
| | PRIMER F3 | FITC | 21 | 23 | 165 |
| | PRIMER F4 | FITC | 24 | 23 | 168 |
| | PRIMER F5 | FITC | 27 | 23 | 171 |
| | PRIMER R | — | 0 | 20 | — |
| MUTANT | PRIMER F | FITC | 36 | 23 | 180 |
| TYPE | PRIMER R | — | 0 | 20 | — |

As shown in Table 1, five different types of wild-type F primers with the mutually different lengths of mobility correction sites can be used in the test area. One type of mutant-type F primer with a longer mobility correction site than the wild-type F primer can be used. Each mobility correction site is formed by DNA. Each labeled site is formed by fluorescein isothiocyanate (FITC) of the same type. These primer sets yield wild-type polynucleotides of 159 to 171 nt and the mutant-type polynucleotides of 180 nt.

Table 2 shows specific examples of the primer sets for the wild-type detection system and the primer sets for the mutant-type detection system for the control area.

TABLE 2

| | | | BASE LENGTH (nt) | | |
|---|---|---|---|---|---|
| | | | MOBILITY | TARGET | |
| PRIMER | | FLUORESCENT | CORRECTION | RECOGNITION | REACTION |
| SET | TYPE | DYE | SITE | SITE | PRODUCT |
| WILD | PRIMER F | FITC | 15 | 23 | 159 |
| TYPE | PRIMER R | FITC | 0 | 20 | — |
| MUTANT | PRIMER F | FITC | 36 | 23 | 180 |
| TYPE | PRIMER R | FITC | 0 | 20 | — |

As shown in Table 2, one type of wild-type F primer is used in the control area. One type of mutant-type F primer with a longer mobility correction site than the wild-type F primer is used. Each mobility correction site is formed by DNA. Each labeled site is formed by fluorescein isothiocyanate (FITC) of the same type. These primer sets yield a wild-type polynucleotide of 159 nt and a mutant-type polynucleotide of 180 nt.

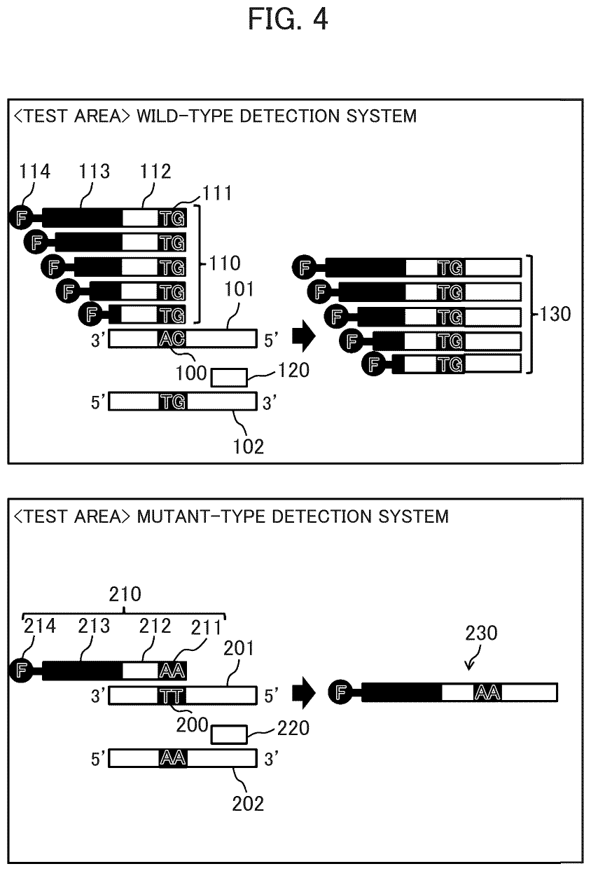

FIG. 4 is a schematic diagram showing the wild-type detection system for detecting the wild-type target base sequence and the mutant-type detection system for detecting the mutant-type target base sequence for the test area. The upper side of FIG. 2 shows the template containing the wild-type target base sequence, the wild-type primer set, and the PCR products amplified by them. The lower part of FIG. 2 shows the template containing the mutant-type target base sequence, the mutant-type primer set, and the PCR products amplified by them.

As shown in FIG. 4, since the wild-type detection system in the test area uses five types of wild-type F primers 110 and R primers 120, performing the PCR amplifies five types of wild-type polynucleotides 130 containing the wild-type target base sequence 100. The wild-type polynucleotides 130 are obtained with the mutually different mobilities in the electrophoresis.

On the other hand, since the mutant-type detection system in the test area uses one mutant-type F primer 210 and R primer 220, performing the PCR amplifies one mutant-type polynucleotide 230 containing the mutant-type target base sequence 200. The mutant-type polynucleotide 230 is obtained with the different mobility in the electrophoresis from the mobility of the wild-type polynucleotide 130.

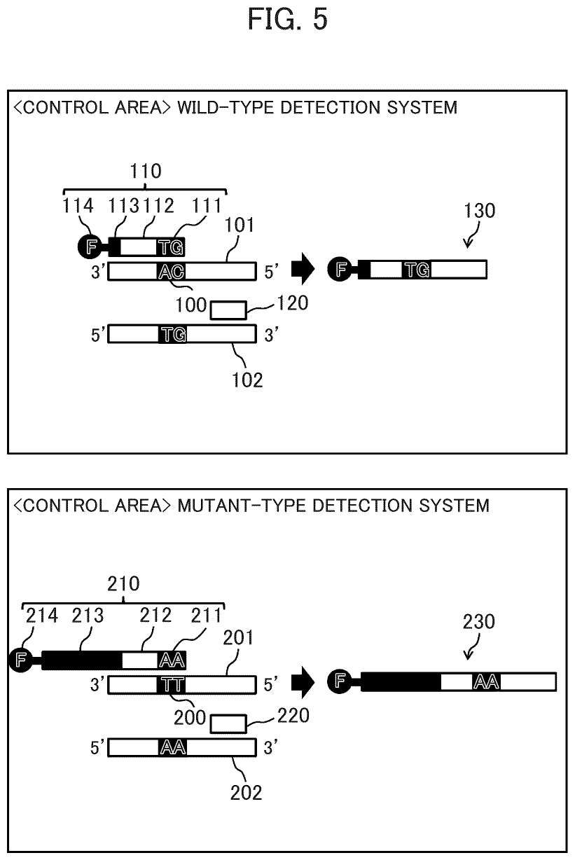

FIG. 5 is a schematic diagram showing the wild-type detection system for detecting the wild-type target base sequence and the mutant-type detection system for detecting the mutant-type target base sequence used in the control area. The upper side of FIG. 5 shows the template containing the wild-type target base sequence, the wild-type primer set, and the PCR products amplified by them. The lower part of FIG. 5 shows the template containing the mutant-type target base sequence, the mutant-type primer set, and the PCR products amplified by them.

As shown in FIG. 5, since the wild-type detection system in the control area uses one wild-type F primer 110 and R primer 120, performing the PCR amplifies one wild-type polynucleotide 130 containing the wild-type target base sequence 100.

On the other hand, since the mutant-type detection system in the test area uses one mutant-type F primer 210 and R primer 220, performing the PCR amplifies one mutant-type polynucleotide 230 containing the mutant-type target base sequence 200.

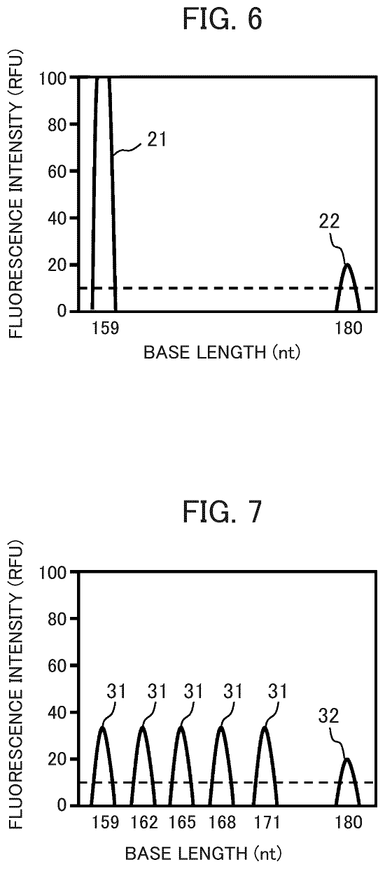

FIG. 6 shows an example of a result of a quantitative analysis in the control area. FIG. 7 shows an example of a result of a quantitative analysis in the test area. FIGS. 6 and 7 show the results of the spectroscopic analysis of fluorescence for each fraction after the PCR was performed using the fluorescent labeling primer sets and the amplified PCR products were fractionated by the electrophoresis. In FIGS. 6 and 7, the horizontal axes indicate the base lengths [nt] of the PCR product. The vertical axes show the fluorescence intensities [cfu] of the PCR products.

Reference sign 21 shows the result of the PCR product amplified by the wild-type primer sets 110, 120 in the control area in which the PCR product is the wild-type polynucleotide 130 containing the relatively large number of wild-type target base sequences 100. Reference sign 22 shows the result of the PCR product amplified by the mutant-type primer sets 210, 220 in the control area in which the PCR product is the mutant-type polynucleotides 230 containing the relatively few mutant-type target base sequences 200. The dashed line shows a specific example of the lower limit of the detection range of the fluorescence intensity.

Reference signs 31 show the result of the PCR products amplified by the wild-type primer sets 110, 120 in the test area in which the PCR products are the wild-type polynucleotides 130 containing the relatively large number of wild-type target base sequences 100. Reference sign 32 shows the results of the PCR products amplified by the mutant-type primer sets 210, 220 in the test area in which the PCR products are the mutant-type polynucleotides 230 containing the relatively few mutant-type target base sequences 200. The dashed line shows a specific example of the lower limit of the detection range of the fluorescence intensity.

As shown in FIG. 6, since one wild-type F primer 110 is used in the control area, the spectroscopic analysis of the fluorescence of each fraction after fractionation of the PCR product by the electrophoresis detects one signal derived from one type of wild-type polynucleotide 130 containing the wild-type target base sequence 100. The signal derived from the wild-type is detected from one fraction, resulting in a larger peak height and a larger peak area.

For example, if the mutation rate is 10% and the fluorescence intensity derived from the mutant-type is 20 RFU, the fluorescence intensity derived from the wild-type is 180 RFU. When attempting to ensure the detection sensitivity for the detection signal derived from the relatively few mutant-type, the detection signal derived from the relatively abundant wild-type may exceed the detection limit of the fluorescence intensity. In such cases, it is difficult to collectively quantify the relatively more abundant wild-type base sequences and the relatively less abundant mutant-type base sequences at the same time.

On the other hand, as shown in FIG. 7, since the plurality of types of wild-type F primers 110 designed to have mutually different mobilities in the electrophoresis are used in the test area, the spectroscopic analysis of the fluorescence of each fraction after the electrophoretic fractionation of the PCR products detects the plurality of signals derived from the plurality of types of wild-type polynucleotides 130 containing the wild-type target base sequence 100. The signals derived from the wild-type are detected in the plurality of fractions and are separated into the plurality of signals, resulting in a smaller peak height and a smaller peak area for each signal.

For example, when the mutation rate is 10% and the fluorescence intensity derived from the mutant-type is 20 RFU, the fluorescence intensity derived from the wild-type is reduced from 180 RFU to 36 RFU. When attempting to ensure the detection sensitivity for the detection signals derived from the relatively few mutant-type, the detection signals derived from the wild-type in the relatively large number can easily be kept within the detection range of fluorescence intensity. Therefore, the proportion of the mutant-type base sequences to the wild-type base sequences can be determined with high accuracy by simultaneously and collectively quantifying the relatively more abundant wild-type base sequences and the relatively less abundant mutant-type base sequences.

### First Embodiment: Reagent for Quantifying Nucleic Acid

The wild-type primer sets 110, 120 and the mutant-type primer sets 210, 220 used for the nucleic acid quantification method according to the first embodiment are nucleic acid quantification reagents for quantifying the proportion of the mutant-type base sequences to the wild-type base sequences in a sample. The reagents can be provided as a kit of predetermined components.

The reagent for quantifying nucleic acid according to the first embodiment includes a plurality of wild-type forward primers (F primers) 110 that are complementary to the wild-type target base sequence 100, the mutant-type forward primers (F primers) 210 that are complementary to the mutant-type target base sequence 200, and the reverse primers (R primer) 120, 220 that are complementary to the complementary strands of the target base sequence 100, 200, as components.

The reagent for quantitative analysis of nucleic acid according to the first embodiment can be provided as a reagent for each analytical target for analysis of any of the target base sequences 100, 200. The wild-type F primer 110, the mutant-type F primer 210, and the R primers 120, 220 are provided in a molecular structure that selectively amplifies polynucleotides containing predetermined target base sequences 100, 200.

The wild-type F primers 110 can include any number of types designed such that the mobilities in the electrophoresis differs among the plurality of wild-type F primers 110. The number of types of wild-type F primers 110 is preferred to be 2 or more and 10 or less, 4 or more and 10 or less is more preferred, and 6 or more and 10 or less is even more preferred. The greater the number of types, the easier it is for the detection signal to fall within the detection range by the detector. However, when the number of types is too large, the reaction rates among the wild-type F primers 110 may be biased, resulting in detection errors.

The mutant-type F primer 210 can include at least one type designed to have a different mobility in the electrophoresis from that of the wild-type F primer 110. The R primers 120, 220 may include at least one type that is common to both the wild type and the mutant type.

In addition to the wild-type F primer 110, the mutant-type F primer 210, and the R primers 120, 220, the reagent for quantifying nucleic acids according to the first embodiment may include one or more of a heat resistant DNA polymerase, a dNTP mixture, and a reaction buffer solution for PCR as components. These components can include the same types as in Step S102 above.

The reagent for quantifying nucleic acid can be accompanied by polynucleotides with the wild-type target base sequence 100 and polynucleotides with the mutant-type target base sequence 200 as components, as a reference for the target base sequences 100, 200 to be analyzed. These references may be attached as genomic DNA or as DNA fragments.

The components of the reagent for quantifying nucleic acid can be provided with each component dissolved in a storage buffer solution and sealed in a container such as a microtube or a microvial. The components of the reagent for quantifying nucleic acid may be enclosed at the concentration at the time of analysis, or they may be enclosed in a more concentrated form than at the time of analysis. The buffer solution in which the polynucleotides are dissolved preferably contains a buffering agent that exhibits a pH buffering effect and chelating agents such as EDTA, and nucleases are preferably inactivated.

The concentrations of the components are not limited. For example, the wild-type F primer 110, the mutant-type F primer 210, the R primers 120, 220, and the reference can be prepared in 0.1 ng/μL or more and 10 ng/μL or less. The DNA polymerase can be prepared in 1 unit/μL or more and 50 units/μL or less. The dNTP mixture or the reaction buffer solution can be prepared with each component in 1 mM or more and 100 mM or less.

The reagent for quantifying nucleic acid according to the first embodiment may be provided either in a state where the labeled sites 114 of the wild-type forward primer 110 and the labeled sites 214 of the mutant-type forward primer 210 are pre-bound, or in an unbound state. The labeled sites 114, 214 can be attached as a component of a reagent for quantifying nucleic acid and can be bound to the mobility correction sites 113, 213, or the like immediately prior to the PCR.

According to the reagent for quantifying nucleic acid according to the first embodiment and the nucleic acid quantification method using the reagent, the PCR using the plurality of types of primers with the mutually different mobilities in the electrophoresis can be performed by a simple operation. Since the plurality of types of polynucleotides derived from the wild-type base sequences with the mutually different mobilities in the electrophoresis, as well as polynucleotides derived from the mutant-type base sequences, can be easily and abundantly prepared in a distinguishable state by a general PCR, the proportion of the mutant-type base sequences to the wild-type base sequences contained in the sample can be quantified quickly and with high accuracy.

### Second Embodiment: Nucleic Acid Quantification Method

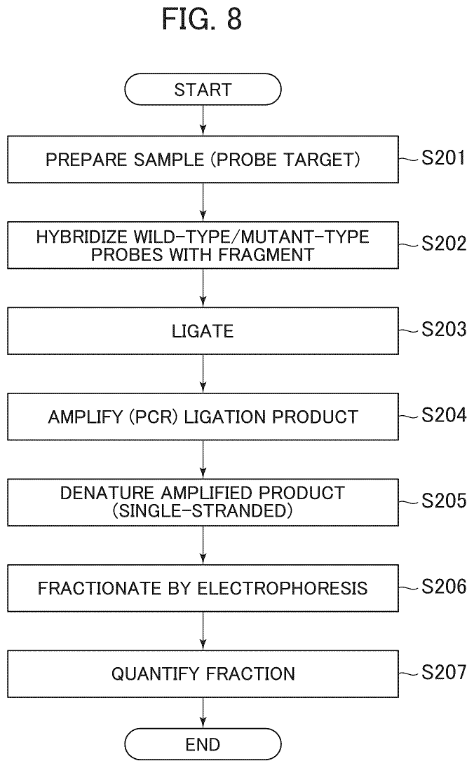

FIG. 8 is a flow diagram showing a nucleic acid quantification method according to the second embodiment of the invention. As shown in FIG. 8, the nucleic acid quantification method according to the second embodiment includes Step S201 of preparing a sample containing a probe target, S202 of hybridizing a probe and a fragment to a target, S203 of ligating the probe and the fragment, S204 of amplifying the ligation product, S205 of denaturing the amplified product, S206 of fractionating the denatured product by electrophoresis, and S207 of quantitatively analyzing the fractionated fractions.

The second embodiment according to the nucleic acid quantification method relates to a method for quantifying the proportion of the mutant-type base sequences to the wild-type base sequences in the sample, similarly to the first embodiment. Examples of sample include a mixture of polynucleotides containing a wild-type base sequence and polynucleotides containing a mutant-type base sequence, such as a nucleic acid solution containing nucleic acids derived from mutually different cells. In this nucleic acid quantification method, the proportion of the mutant type to the wild type is determined for a predetermined target base sequence contained in a sample.

In the nucleic acid quantification method of the second embodiment, the wild-type base sequences and the mutant-type base sequences in the sample are detected by predetermined probes complementary to the respective base sequences. Then, the ligation is performed by the probe set, and the ligated ligation products are fractionated by the electrophoresis to quantify each fraction.

The probe set is comprised of a probe complementary to a target base sequence and a 3′-terminal side of the sequence, and a fragment complementary to an adjacent base sequence on a 5′-terminal side of the target base sequence. The analytical target base sequence is detected by the probe that selectively binds to the base sequence.

In the nucleic acid quantification method according to the second embodiment, a plurality of probes that have complementary base sequences to the wild-type base sequences and mutually different mobilities in the electrophoresis are used as the wild-type probes that recognize the base sequences of the wild-type. As the mutant-type probe that recognizes the mutant-type base sequence, a probe that has a complementary base sequence to the target base sequence of the mutant-type and the mobility in the electrophoresis different from that of the wild-type is used.

The ligation products are fractionated by the electrophoresis, and each fraction is quantified to obtain quantitative results of the ligation products from the wild-type probes and fragments and quantitative results of the ligation products from the mutant-type probes and fragments. Based on these quantitative results, the proportion of the mutant-type base sequence to the wild-type the base sequence can be determined.

### (Step S201)

Step S201 is a step of preparing a sample which is an analytical target for quantifying the proportion of the mutant-type base sequence to the wild-type base sequence and which contains polynucleotides including the target base sequences to be detected by the probes.

The same sample as in Step S101 above can be used as the analytical target. As the polynucleotides containing the target base sequence, single-stranded polynucleotides are preferred from the viewpoint of ensuring the binding efficiency of the probe.

### (Step S202)

Step S202 is a step of hybridizing the probe and the fragment to the target using the target and the probe set.

In Step S202, the wild-type probe set and the mutant-type probe set are hybridized to a wild-type polynucleotide containing the target wild-type base sequence and a mutant-type polynucleotide containing the target mutant-type target base sequence, respectively. In Step S202, the wild-type target base sequence is detected by the wild-type probes that constitute the wild-type probe set. The mutant-type target base sequence is also detected by the mutant-type probes that constitute the mutant-type probe set.

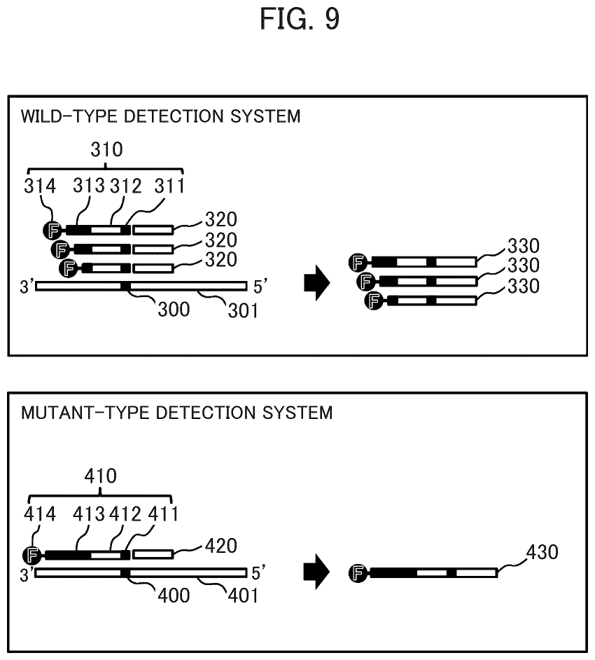

FIG. 9 is a schematic diagram showing a wild-type detection system for detecting wild-type target base sequences and a mutant-type detection system for detecting mutant-type target base sequences. The upper side of FIG. 9 shows the target containing the wild-type target base sequence, the wild-type probe set, and the ligation products produced by them. The lower part of FIG. 9 shows the target containing the mutant-type target base sequence, the mutant-type probe set, and the ligation products produced by them.

As shown in the upper part of FIG. 9, a wild-type polynucleotide (a target strand) 301, which contains the wild-type target base sequence 100 included in the sample, is the target of the probe in the wild-type detection system. The wild-type detection system, which detects the wild-type target base sequences, is composed of wild-type probe set 310, 320. The wild-type probe set 310, 320 are composed of a plurality of wild-type probes 310 and fragments 320.

The wild-type probe 310 binds to the target strand 301 containing a wild-type target base sequence 300. The wild-type probe 310 has a complementary base sequence to the target strand 301. The wild-type probe 310 is composed of a plurality of types designed to be mutually different in the mobility in the electrophoresis. The plurality of wild-type probes 310 each selectively bind to the region containing the wild-type target base sequence 300 of each target strand 301 included in the sample.

The fragment 320 binds to the target strand 301, which contains the wild-type target base sequence 300. The fragment 320 has a sequence complementary to target strand 301. The fragment 320 selectively binds to an adjacent base sequence on a 5′-terminal side of the target base sequence 300 so as to be adjacent to the wild-type probe 310. The fragment 320 is preferably phosphorylated at the 5′-terminal side for ligation.

As shown in the lower part of FIG. 9, a mutant-type polynucleotide (target strand) 401, which contains a mutant-type target base sequence 400 contained in the sample, is the target of the probe in the mutant-type detection system. The mutant-type detection system for detecting the mutant-type target base sequences is composed of mutant-type probe set 410, 420. The mutant-type probe set 410, 420 include a mutant-type probe 410 and a fragment 420.

The mutant-type probe 410 binds to the target strand 401 containing the mutant-type target base sequence 400. The mutant-type probe 410 has a complementary base sequence to the target strand 401. The mutant-type probe 410 is composed of at least one type designed to have a different mobility in the electrophoresis from that of the wild-type probe 310. The mutant-type probe 410 selectively binds to the region containing the mutant-type target base sequence 400 of each target strand 401 included in the sample.

The fragment 420 binds to the target strand 401, which contains the mutant-type target base sequence 400. The fragment 420 has a sequence complementary to target strand 401. The fragment 420 selectively binds to the adjacent base sequence on the 5′-terminal side of target base sequence 400, so as to be adjacent to the mutant-type probe 410. The fragment 420 is preferred to be phosphorylated at the 5′-terminal side for ligation.

In Step S202, the wild-type detection system and the mutant-type detection system are reacted in the same reaction system. To the same reaction solution in which the hybridization is performed, add a plurality of wild-type probes 310, a mutant-type probe 410, and at least one fragment 410, 420, which are mutually different in the mobilities in the electrophoresis, in substantially equal amounts to one another. At least one type of fragment 410, 420 can be added for both the wild-type and the mutant-type.

The hybridization can be performed by adding the target polynucleotide, the wild-type probe set 310, 320, and the mutant-type probe set 410, 420 to the reaction buffer solution and following the usual methods. As a reaction buffer solution, an aqueous solution in which a buffer agent that shows a pH buffering effect, a denaturant that denatures polynucleotides, and additives that are added as necessary can be used.

Examples of the buffering agents include sodium chloride-acetate buffer, Tris-HCl buffer, Tris-acetate buffer, HEPES buffer, and phosphate buffer. Denaturants include, for example, formamide and urea. Examples of the additives include surfactants such as TritonX-100, Tween 20, and B SA.

The reaction conditions for hybridization is preferred to be, for example, 50° C. or higher and 60° C. or lower for 30 seconds or more and 60 seconds or less. The reaction conditions for hybridization can be appropriately adjusted according to the base length and GC content of the probes and the fragments. The reaction conditions for hybridization include the concentrations of the target, the wild-type probe set 310, 320, the mutant-type probe set 410, 420, and the additives, as well as temperature and time.

As shown in FIG. 9, the wild-type probe 310 has a mutation recognition site 311, a target recognition site 312, a mobility correction site 313, and a labeled site 314. The mutation recognition site 311, the target recognition site 312, the mobility correction site 313 and the labeled site 314 are arranged in a state of being connected in this order from the 3′-terminal side of the wild-type probe 310.

The mutant-type probe 410 has a mutation recognition site 411, a target recognition site 412, a mobility correction site 413, and a labeled site 414. The mutation recognition site 411, the target recognition site 412, the mobility correction site 413, and the labeled site 414 are arranged in a state of being connected in this order from the 3′-terminal side of the mutant-type probe 410.

The mutation recognition sites 311, 411 are sites that recognize the target base sequence 300, 400 and identify presence or absence of mutations in the target base sequence 300, 400. The mutation recognition sites 311, 411 can be formed with polynucleotides of any degree of polymerization.

The mutation recognition site 311 of the wild-type probe 310 is a complementary base sequence to the wild-type target base sequence 300. The mutation recognition site 311 of the wild-type probe 310 is provided in a common base sequence among the plurality of wild-type probes 310.

The mutation recognition site 411 of the mutant-type probe 410 is a complementary base sequence to the mutant-type target base sequence 400. The mutation recognition site 411 of the mutant-type probe 410 is preferred to be formed of the same base sequence as the mutation recognition site 311 of the wild-type probe 310, except for the loci complementary to the mutated loci. Such a base sequence facilitates the adjustment of the mobility in the electrophoresis.

According to the mutation recognition sites 311, 411, it is identified whether the target base sequence of the polynucleotide contained in the sample is the wild-type base sequence or the mutant-type base sequence. When the mutation recognition site 311 of the wild-type probe 310 is easy to bind and the ligation product generated by the wild-type probe set 310, 320 is relatively large, the target base sequence in question is the wild type. On the other hand, when the mutation recognition site 411 of the mutant-type probe 410 is easy to bind and the ligation product generated by the mutant-type probe set 410, 420 is relatively large, the target base sequence in question is the mutant-type.

The lengths of mutation recognition sites 311, 411 are not particularly limited, but 1 nt or more and 6 nt or less is preferred, and 1 nt or more and 5 nt or less is more preferred. Such a length increases the efficiency of the selective hybridization and thus reduces the misidentification of the wild type and the mutant type.

The mutation recognition sites 311, 411 are provided at the 3′-terminal sides of the respective probes 310, 410. The nucleotide at the 3′-terminal side of each of the probes 310, 410 is preferred to be in an arrangement that forms a hydrogen bonding with a base that produces the polymorphism due to mutation in the target base sequences 300, 400. This arrangement makes the ligation less likely to occur when the mutation recognition sites 311, 411 are mishybridized. Artifacts due to the mishybridization are reduced, allowing accurate identification of the wild type and the mutant type.

The target recognition sites 312, 412 are sites for recognizing common sequences other than the target base sequences 300, 400 to identify the bonding positions in the hybridization. The target recognition sites 312, 412 are connected to the 5′-terminal side of the mutation recognition sites 311, 411. The target recognition sites 312, 412 can be formed with polynucleotides of any degree of polymerization.

The target recognition sites 312, 412 are complementary base sequences to the common sequence adjacent to the target base sequences 300, 400. The common sequence is the base sequence that is common to the target strand 301 with the wild-type target base sequence 300 and the target strand 401 with the mutant-type target base sequence 400.

The target recognition site 312 of the wild-type probe 310 is provided in a common sequence among the plurality of wild-type probes 310. The target recognition site 412 of the mutant-type probe 410 is preferred to be formed by the same base sequence as the target recognition site 312 of the wild-type probe 310. Such a base sequence facilitates the adjustment of the mobility in the electrophoresis.

With the target recognition sites 312, 412, the target strands 301, 401 with the target base sequences 300, 400 contained in the sample and the bonding positions in the hybridization are identified. Since the position-selective bonding of each of the probes 310, 410 to the target is secured, the target base sequence 300, 400 can be identified by the mutation recognition sites 311, 411, and can be ligated with fragments 320, 420, properly.

The lengths of the target recognition sites 312, 412 are not particularly limited, but 10 nt or more and 50 nt or less is preferred, and 10 nt or more and 30 nt or less is more preferred. Such a length increases the efficiency of the selective hybridization to the target strands 301, 401, thus reducing the target misidentification and the misidentification of the bonding positions.

The mobility correction sites 313, 413 are sites for correcting the mobilities in the electrophoresis for quantitative target polynucleotides 330, 430, which are the ligation products. The mobility correction sites 313, 413 are connected to the 5′-terminal side of the target recognition sites 312, 412. The mobility correction sites 313, 413 can be formed with polynucleotides of any degree of polymerization or polymers that are non-polynucleotides of any degree of polymerization.

The mobility correction site 313 of the wild-type probe 310 is provided in a molecular structure where the mobilities in the electrophoresis differ among the plurality of wild-type probes 310. The mobility correction site 413 of the mutant-type probe 410 is provided in a molecular structure with the mobility in the electrophoresis different from that of the mobility correction site 313 of the wild-type probe 310.

With the mobility correction sites 313, 413, the ligation using each of the probe sets 310, 320, 410, 420 can synthesize the plurality of types of polynucleotides 330, 430 with the mutually different mobilities in the electrophoresis. When the ligation products are fractionated into fractions by the electrophoresis and the quantification is performed by detecting the labeling of each fraction, the wild-type polynucleotide 330 containing a relatively large number of the wild-type target base sequences 300 can be divided into the plurality of fractions. Since the detection signal derived from the wild type is subdivided into fractions, the detection signal derived from the wild type can be easily brought within the detection range by the detector. The difference in the mobility in the electrophoresis between wild-type and the mutant-type allows easily distinguishing between the relatively more abundant wild-type and the relatively less abundant mutant-type.

The mobility correction sites 313, 413 can be provided in different molecular lengths, molecular weights, molecular structures, and the like such that the mobilities in the electrophoresis mutually differs from one another among the plurality of wild-type probes 310 or between the wild-type probes 310 and the mutant-type probes 410.

The mobility correction sites 313, 413 are preferably provided in a molecular structure with a common part among the plurality of wild-type probes 310 and between the wild-type probes 310 and the mutant-type probes 410. The mobility in the electrophoresis is preferred to be adjusted by an additional part connected to the common part. The common part is preferred to be provided on the side of the target recognition sites 312, 412. Such a molecular structure allows for easy adjustment of the mobilities in the electrophoresis while ensuring selective binding to the target base sequences 300, 400. The mobility correction sites 313, 413, similarly to the mobility correction sites 113, 213 described above, may be formed solely of polynucleotides, solely of non-polynucleotide polymers, or a combination of these. It may be provided in a linear molecular chain or in a branched molecular structure.

The lengths of mobility correction sites 313, 413 are not particularly limited when formed by polynucleotides, but 10 nt or more and 100 nt or less is preferred. Such a length reduces the mishybridization of each of the probes 310, 410 through the mobility correction sites 313, 413.

When formed with polynucleotides, the mobility correction sites 313, 413 can be set to an appropriate degree of polymerization difference of at least 1 nt or more degree of polymerization difference between the plurality of wild-type probes 310 or between the wild-type probe 310 and the mutant-type probe 410. A polymerization degree difference of 5 nt or more is preferred from the standpoint of separability in electrophoresis, and 10 nt or more is more preferred.

The length of the mobility correction site 413 of the mutant-type probe 410 is preferred to be longer than the length of the mobility correction site 313 of the wild-type probe 310. In general, the longer the molecular chain, the more likely the electrophoresis mobility is to vary and the more likely it is to produce noise in the detection signal. In contrast, when the mobility correction site 413 of the probe 410 for the mutant-type is relatively long, the relatively short ligation product amplified by the wild-type probe 310 will be less noisy. Accurate quantitation can be performed because noise mixing due to the plurality of types of ligation products can be avoided.

The labeled sites 314, 414 are sites for labeling the ligation product, the quantitative target polynucleotide. The labeled sites 314, 414 can be formed with the fluorescent dyes, the radioactive labels, or the like. The labeled sites 314, 414 are preferred to be connected to the terminal side of the mobility correction sites 313, 413 opposite to the target recognition sites 312, 412 when the fluorescent dyes are used.

With the labeled sites 314, 414, the ligation using each of the probe sets 310, 320, 410, 420 can yield the wild-type polynucleotides 330 and the mutant-type polynucleotides 430 labeled with fluorescent dyes, radioisotopes, and the like. When the ligation products are fractionated into fractions by the electrophoresis, the label of each fraction can be detected and quantified.

The same types of fluorescent dyes and radioisotopes that form the labeled sites 314, 414 can be used as the labeled sites 114, 214 described above. The labeled site 314 of the wild-type probe 310 may be labeled with a fluorescent dye that emits fluorescence at mutually different wavelengths or the same wavelength among the plurality of wild-type probes 310. The labeled site 314 of the wild-type probe 310 may be labeled with a fluorescent labeling dye that emits fluorescence at the different wavelengths from the labeled site 414 of the mutant-type probe 410, or they may be labeled with fluorescent dyes that emit fluorescence at the same wavelength.

The lengths of the fragments 320, 420 are not particularly limited, but 10 nt or more and 50 nt or less is preferred, and 10 nt or more and 30 nt or less is more preferred. Such a length increases the efficiency of the selective hybridization to the target strand complementary strands 301, 401, thus reducing the misidentification of the target and the misidentification of the ligation position.

### (Step S203)

Step S203 is a step of ligating the probe and the fragment hybridized on the target to one another.

In Step S203, the wild-type probe 310 and the fragment 320 hybridized to the target polynucleotide (a target strand 301) containing the wild-type target base sequence 300 are ligated to one another by a ligase, and the mutant-type probe 410 and the fragment 420 hybridized to the target polynucleotide (a target strand 401) containing the mutant-type target base sequence 400 are ligated to one another by the ligase. Since only the probes 310, 410 that hybridize properly are substrates for the ligase, the wild-type target base sequence and the mutant-type target base sequence can be distinguished.

The ligation can be performed by dissolving the wild-type probe 310 and the fragment 320 hybridized to the target strand 301, the mutant-type probe 410 and the fragment 420 hybridized to the target strand 401, and the ligase in a reaction buffer solution according to the usual method. As the reaction buffer solution, an aqueous solution containing a buffering agent that exhibits a pH buffering effect, a cofactor magnesium ion, ATP, a reducing agent such as dithiothreitol, and additives added as necessary can be used.

Examples of the ligases include T4 DNA ligase and TaqDNA ligase. Examples of the buffering agents include Tris-EDTA buffer. Examples of the additives include molecular crowding promoters that promote molecular association, such as polyethylene glycol, dextran, and albumin. The addition of molecular crowding promoters can increase the binding rate between the probes and the fragments because the high concentration of molecules limits the reaction field and improves the activity.

Reaction conditions for the ligation are preferred to be, for example, 16° C. or higher and 42° C. or lower for at least 30 minutes. The reaction conditions for the ligation can be appropriately adjusted according to the base length and the concentrations of the probes and the fragments. The reaction conditions for the ligation include the concentrations of the target, the wild-type probe set 310, 320, the mutant-type probe set 410, 420, the ligase, and the additives, as well as temperature and time.

### (Step S204)

Step S204 is a step of amplifying polynucleotides containing the target base sequence which is the ligation product, by PCR using a template and a primer set. Step S204 may be omitted when a large amount of ligation product suitable for quantitation is obtained.

In Step S204, the PCR using a predetermined primer set is used to amplify the ligation product, that is, the wild-type polynucleotide 330 containing the wild-type target base sequence 300, and the mutant-type polynucleotide 430 containing the mutant-type target base sequence 400. In Step S204, a large amount of polynucleotides suitable for quantitation is synthesized by the amplification of the ligation products.

The PCR can be performed as in Step S102, by adding the template polynucleotides 330, 430, the wild-type primer set, the mutant-type primer set, a DNA polymerase with heat resistance, and a dNTP mixture to the reaction buffer solution according to the usual method. As for the PCR, a common thermal cycle reaction can be used.

As the wild-type primer set, a combination of a forward primer complementary to an adjacent base sequence adjacent to a 5′-terminal side of the complementary base sequence complementary to fragment 320 and a reverse primer complementary to an adjacent base sequence adjacent to a 3′-terminal side of the complementary base sequence complementary to the mobility correction site 313 can be used.

As the mutant-type primer set, a combination of a forward primer complementary to an adjacent base sequence adjacent to a 5′-terminal side of the complementary base sequence complementary to fragment 420 and a reverse primer complementary to an adjacent base sequence adjacent to a 3′-terminal side of the complementary base sequence complementary to the mobility correction site 413 can be used.

When amplifying the ligation product by the PCR, a primer labeled with the same labeled site as the labeled sites 314, 414 can be used as the forward primer. In such a case, the labeled sites 314, 414 need not be connected to each of the probes 310, 410.

### (Step S205)

Step S205 is a step of preparing a sample for electrophoresis by denaturing the ligation product produced by ligation or the polynucleotide as an amplified product amplified by the PCR after the ligation into the single strands.

In Step S205, the wild-type polynucleotide 330 containing the wild-type target base sequence 300 produced by ligation, and the mutant-type polynucleotide 430 containing the mutant-type target base sequence 400 produced by ligation, or the PCR products amplified from these, are dissociated into the single strands suitable for the electrophoresis. The ions and other substances in the reaction solution are separated or diluted to prepare the sample suitable for electrophoresis.

Denaturation of the polynucleotides can be performed in the same manner as in Step S103, using a general method such as chemical treatment, heat treatment, or a combination thereof, or using a commercially available purification kit including a purification column. The sample for the electrophoresis preferably contains, in addition to the amplified polynucleotides 330, 430, a buffering agent that exhibits a pH buffering effect and a chelating agent such as EDTA. The samples for the electrophoresis is preferred to be adjusted to pH 7.5 or more and pH 8.5 or less. Tris-acetate buffer and Tris-borate buffer are preferred buffering agents.

### (Step S206)

Step S206 is a step of fractionating the ligation product produced by ligation or the amplified product amplified by the PCR after the ligation, which is a polynucleotide, by the electrophoresis.

In Step S206, the wild-type polynucleotide 330 containing the wild-type target base sequence 300 produced by ligation, the mutant-type polynucleotide 430 containing the mutant-type target base sequence 400 produced by ligation, or the PCR products amplified from these are separated into fractions according to molecular weight by the electrophoresis.

The electrophoresis of polynucleotides can be performed by capillary electrophoresis, gel electrophoresis, or the like as in Step S104. The electrophoresis of the polynucleotides is preferably performed by capillary electrophoresis from the viewpoint of the high resolution and quantitative and the collective loading and detection of the PCR products. From the viewpoint of utilizing the molecular sieving effect, it is more preferable to perform capillary gel electrophoresis using gel as the separation media.

### (Step S207)

Step S207 is a step of performing a quantitative analysis of the fractions fractionated by the electrophoresis.

In Step S207, the wild-type polynucleotide 330 containing the wild-type target base sequence 300 fractionated by molecular weight using the electrophoresis, and the mutant-type polynucleotide 430 containing the mutant-type target base sequence 400, or the PCR products amplified from these, are quantified for each fraction fractionated by the electrophoresis, and the proportion of the mutant-type base sequence to the wild-type base sequence is determined.

The proportion of the mutant-type base sequence to the wild-type base sequence can be derived in the same manner as in Step S105, by dividing the amount of the mutant-type polynucleotide by a sum of an amount of the wild-type polynucleotide and an amount of the mutant-type polynucleotide based on the quantitative results of the wild-type polynucleotide and the mutant-type polynucleotide.

Here, the quantitative method for quantifying the proportion of the mutant-type base sequences to the wild-type base sequences is described more specifically for genomic DNA.

As the polynucleotide containing the wild-type target base sequence, genomic DNA BRAF Wild Type Reference Standard (manufactured by Horizon Discovery) having the wild-type BRAF gene is to be used, as in the analysis example using the above-described PCR. The polynucleotide containing the mutant-type target base sequence is to be genomic DNA BRAFV600K (manufactured by Horizon Discovery) having the mutant-type BRAF gene.

In the quantitative analysis, first, the wild-type polynucleotide containing the wild-type target base sequence and the mutant-type polynucleotide containing the mutant-type target base sequence are generated by ligation using the wild-type probe set and the mutant-type probe set. The polynucleotide as the amplified product is then fractionated by the electrophoresis, and the fractionated fractions are subjected to the quantitative analysis to determine the proportion of the mutant-type base sequence to the wild-type base sequence.

Table 3 shows specific examples of probe sets for the wild-type detection system and the mutant-type detection system.

TABLE 3

| | | | BASE LENGTH (nt) | | |
|---|---|---|---|---|---|
| | | | MOBILITY | TARGET | |
| PROBE | | FLUORESCENT | CORRECTION | RECOGNITION | REACTION |
| SET | TYPE | DYE | SITE | SITE | PRODUCT |
| WILD | PROBE L1 | FITC | 15 | 23 | 159 |
| TYPE | PROBE L2 | FITC | 18 | 23 | 162 |
| | PROBE L3 | FITC | 21 | 23 | 165 |
| | PROBE L4 | FITC | 24 | 23 | 168 |
| | PROBE L5 | FITC | 27 | 23 | 171 |
| | PROBE R | — | 0 | 20 | — |
| MUTANT | PROBE L | FITC | 36 | 23 | 180 |
| TYPE | PROBE R | — | 0 | 20 | — |

As shown in Table 3, the five types of wild-type probes with different lengths of the mobility correction sites can be used in the test area. As the mutant-type probe, one type with a longer mobility correction site than the wild-type probes can be used. Each mobility correction site is formed by DNA. Each labeled site is formed by fluorescein isothiocyanate (FITC) of the same type. With these probes, the wild-type polynucleotide of 159 to 171 nt and the mutant-type polynucleotide of 180 nt can be obtained.

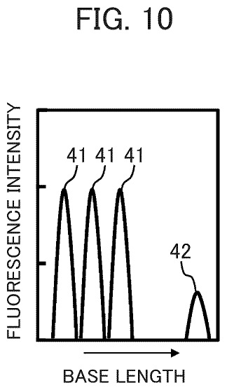

FIG. 10 shows an example of a result of a quantitative analysis of fractions fractionated by the electrophoresis. FIG. 10 shows the result of a spectroscopic analysis of fluorescence for each fraction after performing the ligation using a fluorescently labeled probe set and fractionating the ligated products by the electrophoresis. In FIG. 4, the horizontal axis indicates the base length of the ligation product. The vertical axis shows the fluorescence intensity of the ligation product.

Reference signs 41 are ligation products ligated by the wild-type probe set 310, 320, and indicates the result of the wild-type polynucleotide 330 containing the relatively abundant wild-type target base sequence 300. Reference sign 42 is a ligation product ligated by the mutant-type probe set 410, 420, and indicates the result of the mutant-type polynucleotide 430 containing the relatively few mutant-type target base sequences 400.

As shown in FIG. 10, the ligation product ligated by the wild-type probe set 310, 320 generates the plurality of detection signals by using the plurality of wild-type probes 310. When the plurality of wild-type probes 310 with the mutually different mobilities in the electrophoresis are used, the detection signal derived from the wild-type target base sequence 300 is divided into the plurality of small signals. Therefore, the detection signal for each fraction can be easily within the lower limit of the detection range, which is above the detection sensitivity, and below the upper limit, which is the detection limit.

In addition, as shown in FIG. 10, the ligation products ligated by the mutant-type probe set 410, 420 generate signals different from those amplified by the wild-type probe set 310, 320. When the mutant-type probe 410 with the mobility different from that of the wild-type probe 310 in the electrophoresis is used, the detection signal derived from the wild-type target base sequence 300 can be distinguished from the detection signal derived from the mutant-type target base sequence 400, thus enabling accurate quantification.

### Second Embodiment: Reagent for Quantifying Nucleic Acid

The wild-type probe set 310, 420 and the mutant-type probe set 410, 420 used in the nucleic acid quantification method according to the second embodiment are nucleic acid quantification reagents for quantifying the proportion of the mutant-type base sequences to the wild-type base sequences contained in the sample, and can be provided as a reagent kit-packaged with predetermined components.

The reagent for quantifying nucleic acid according to the second embodiment includes the plurality of wild-type probes 310 complementary to the wild-type target base sequence 300, the mutant-type probes 410 complementary to the mutant-type target base sequence 400, and the fragments 320, 420 complementary to the adjacent base sequences adjacent to the 5′-terminal side of the target base sequences 300, 400.

The reagent for quantifying nucleic acid according to the second embodiment can be provided as a reagent for each analytical target, with arbitrary target base sequences 300, 400 as the analytical targets. The wild-type probe 310, the mutant-type probe 410, and the fragments 320, 420 are provided to a molecular structure that selectively hybridizes with polynucleotides containing the predetermined target base sequences 300, 400.

The wild-type probe 310 may include any number of types designed such that the mobilities in the electrophoresis differs between the plurality of wild-type probes 310. The number of types of the wild-type probes 310 is preferably two or more and ten or less, more preferably four or more and ten or less, and even more preferably six or more and ten or less. The greater the number of types, the easier it is for the detection signal to fall within the detection range by the detector. However, when the number of types is too large, the reaction rate between the wild-type probes 310 may become uneven, resulting in detection errors.

The mutant-type probe 410 may include at least one type designed such that its mobility in the electrophoresis is different from that of the wild-type probe 310. The fragments 320, 420 may include at least one type that is common to both the wild type and the mutant type.

The nucleic acid quantification reagent according to the second embodiment may contain, in addition to the wild-type probe 310, the mutant-type probe 410, and the fragments 320, 420, one or more of ligase, ATP, and a reaction buffer solution for ligation as components. These components may include the same types as those described in Step S203 above.

The reagent for quantifying nucleic acid may also include, as a reference for the target base sequence 300, 400 of the analytical target, a polynucleotide having a wild-type target base sequence 300 or a polynucleotide having a mutant-type target base sequence 400 as a constituent. These references may be attached as genomic DNA or as DNA fragments.

The components of the reagent for quantifying nucleic acid can be provided, with each component dissolved in a storage buffer solution and sealed in a container such as a microtube or a microvial. The components of the reagent for quantifying nucleic acid may be enclosed at the concentration at the time of analysis, or they may be enclosed in a more concentrated form than at the time of analysis. The buffer solution in which the polynucleotides are dissolved preferably contains a buffering agent that exhibits a pH buffering effect and chelating agents such as EDTA, and nucleases are preferably inactivated.

The concentrations of the components are not limited. For example, the wild-type probe 310, the mutant-type probe 410, the fragments 320, 420, and the reference can be prepared at 0.1 ng/μL or more and 10 ng/μL or less. The ligase can be prepared at a concentration of 1 unit/μL or more and 50 units/μL or less. The reaction buffer solution can be prepared such that each component is 1 mM or more and 100 mM or less.

The reagent for quantifying nucleic acid according to the second embodiment may be provided with the labeled site 314 of the wild-type probe 310 and the labeled site 414 of the mutant-type probe 410 in a pre-bound state or in an unbound state. The labeled sites 314, 414 can be attached as components of the reagent for quantifying nucleic acid and bound to the mobility correction sites 313, 413, or the like immediately before PCR.

With the nucleic acid quantification reagent of the second embodiment and the nucleic acid quantification method using the same, the ligation using the plurality of types of probes with the different mobilities in the electrophoresis can be performed by a simple operation. Since the plurality of types of polynucleotides derived from the wild-type base sequences with the different mobilities in the electrophoresis and the polynucleotides derived from the mutant-type base sequences can be easily prepared in a mutually distinguishable state by a general ligation, the proportion of the mutant-type base sequences to the wild-type base sequences contained in the sample can be quantified quickly and with high accuracy.

The above describes the present invention, but the present invention is not limited to the above-described embodiments, and various modifications may be made within the scope of the present invention without departing from the spirit and scope of the present invention. For example, the present invention is not limited to those having all the configurations provided in the above-described embodiments. It is possible to replace a part of the configuration of one embodiment with another configuration, add a part of the configuration of one embodiment to another configuration, or omit a part of the configuration of one embodiment.

A nucleic acid quantification method using the above-described PCR may include: a step of amplifying wild-type polynucleotides containing wild-type target base sequences and mutant-type polynucleotides containing mutant target base sequences by PCR using a wild-type primer set including a plurality of wild-type forward primers complementary to the wild-type target base sequence and a reverse primer complementary to a complementary strand of the target base sequence, and a mutant-type primer set including a mutant-type forward primer complementary to a mutant target base sequence and a reverse primer complementary to a complementary strand of the target base sequence; a step of fractionating the wild-type polynucleotides and the mutant-type polynucleotides by electrophoresis: a step of detecting a label of the wild-type polynucleotide to quantify the wild-type polynucleotide and detecting a label of the mutant-type polynucleotide and to quantify the mutant-type polynucleotide; and a step of determining the proportion of the mutant-type polynucleotide to the wild-type polynucleotide based on the quantitative result of the wild-type polynucleotide and the quantitative result of the mutant-type polynucleotide.

A nucleic acid quantification method using the above-described ligation may include: a step of hybridizing a wild-type probe set including of a plurality of wild-type probes complementary to a wild-type target base sequence and a fragment complementary to an adjacent base sequence adjacent to a 5′-terminal side of the target base sequence and a mutant-type probe set including a mutant-type probe complementary to a mutant-type target base sequence and a fragment complementary to an adjacent base sequence adjacent to a 5′-terminal side of the target base sequence, to a wild-type polynucleotide containing the wild-type target base sequence and a mutant-type polynucleotide containing the mutant-type target base sequence; a step of producing a wild-type polynucleotide including the wild-type target base sequence by ligating the wild-type probe and the fragment hybridized to the wild-type polynucleotide and producing a wild-type polynucleotide including the wild-type target base sequence and by ligating the mutant-type probe and the fragment hybridized to the mutant-type polynucleotide; a step of fractionating the wild-type polynucleotides and the mutant-type polynucleotides by electrophoresis: a step of detecting a label of the wild-type polynucleotide to quantify the wild-type polynucleotide and detecting a label of the mutant-type polynucleotide and to quantify the mutant-type polynucleotide; and a step of determining the proportion of the mutant-type polynucleotide to the wild-type polynucleotide based on the quantitative result of the wild-type polynucleotide and the quantitative result of the mutant-type polynucleotide.

In addition, the nucleic acid quantification method using the above-described ligation may include: a step of hybridizing a wild-type probe set including a plurality of wild-type probes complementary to a wild-type target base sequence and a fragment complementary to an adjacent base sequence adjacent to a 5′-terminal side of the target base sequence and a mutant-type probe set including a mutant-type probe complementary to a mutant-type target base sequence and a fragment complementary to an adjacent base sequence adjacent to a 5′-terminal side of the target base sequence, to a wild-type polynucleotide containing the wild-type target base sequence and a mutant-type polynucleotide containing the mutant-type target base sequence; a step of producing a wild-type polynucleotide including the wild-type target base sequence by ligating the wild-type probe and the fragment hybridized to the wild-type polynucleotide and producing a wild-type polynucleotide including the wild-type target base sequence and by ligating the mutant-type probe and the fragment hybridized to the mutant-type polynucleotide; a step of amplifying the wild-type polynucleotide and the mutant-type polynucleotide by PCR; a step of fractionating the amplified wild-type polynucleotides and the amplified mutant-type polynucleotides by electrophoresis: a step of detecting a label of the wild-type polynucleotide to quantify the wild-type polynucleotide and detecting a label of the mutant-type polynucleotide and to quantify the mutant-type polynucleotide; and a step of determining the proportion of the mutant-type polynucleotide to the wild-type polynucleotide based on the quantitative result of the wild-type polynucleotide and the quantitative result of the mutant-type polynucleotide.

### LIST OF REFERENCE SIGNS

100, 200, 300, 400: Target base sequence,
101, 201, 301, 401: Target strand,
102, 202: Complementary strand,
110: Wild-type forward primer (F primer)
210: Mutant-type forward primer (F primer),
120, 220: Reverse primer (R primer),
130: Wild-type polynucleotides (PCR product),
230: Mutant-type polynucleotide (PCR product),
310: Wild-type probe,
410: Mutant-type probe,
320,420: Fragment,
330: Wild-type polynucleotide (ligation product),
430: Mutant-type polynucleotide (ligation product),
111, 211, 311, 411: Mutation recognition site
112, 212, 312, 412: Target recognition location
113, 213, 313, 413: Mobility correction site
114, 214, 314, 414: Labeled site

1. A nucleic acid quantification method for quantifying a proportion of a mutant-type base sequence to a wild-type base sequence contained in a sample, comprising:
a step of amplifying wild-type polynucleotides containing wild-type target base sequences and mutant-type polynucleotides containing mutant-type target base sequences by PCR using a wild-type primer set including a plurality of wild-type forward primers complementary to the wild-type target base sequence and a reverse primer complementary to a complementary strand of the target base sequence, and a mutant-type primer set including a mutant-type forward primer complementary to a mutant target base sequence and a reverse primer complementary to a complementary strand of the target base sequence; and
a step of fractionating the wild-type polynucleotides and the mutant-type polynucleotides by electrophoresis to determine the proportion of the mutant-type polynucleotides to the wild-type polynucleotides, wherein
the wild-type forward primer has a base sequence complementary to the wild-type target base sequence at a 3′-terminal side,
the mutant-type forward primer has a base sequence complementary to the mutant-type target base sequence at the 3′-terminal side, and
the plurality of wild-type forward primers have mutually different molecular structures that result in different mobilities in the electrophoresis.

2. The nucleic acid quantification method according to claim 1, wherein
the wild-type forward primer has a mobility correction site that corrects a mobility in the electrophoresis, and
the mobility correction site is formed by a polynucleotide.

3. The nucleic acid quantification method according to claim 1, wherein
the wild-type forward primer has a mobility correction site that corrects a mobility in the electrophoresis, and
the mobility correction site is formed by a non-polynucleotide polymer.

4. The nucleic acid quantification method according to claim 3, wherein
the polymer as the non-polynucleotide has a molecular structure with a branch.

5. The nucleic acid quantification method according to claim 1, wherein
the wild-type forward primer and the mutant-type forward primer are labeled with respective fluorescent dyes.

6. The nucleic acid quantification method according to claim 5, wherein
the plurality of wild-type forward primers are labeled with respective fluorescent dyes that emit fluorescence at mutually different wavelengths.

7. The nucleic acid quantification method according to claim 5, wherein
the plurality of wild-type forward primers are labeled with fluorescent dyes that emit fluorescence at mutually different wavelengths being different from a wavelength of the mutant-type forward primer.

8. The nucleic acid quantification method according to claim 1, wherein
the wild-type forward primer and the mutant-type forward primer are labeled with respective radioisotopes.

9. A nucleic acid quantification method for quantifying a proportion of a mutant-type base sequence to a wild-type base sequence contained in a sample, comprising:
a step of hybridizing, to a wild-type polynucleotide containing the wild-type target base sequence and a mutant-type polynucleotide containing the mutant-type target base sequence, a wild-type probe set including a plurality of wild-type probes complementary to a wild-type target base sequence and a fragment complementary to an adjacent base sequence adjacent to a 5′-terminal side of the target base sequence and a mutant-type probe set including a mutant-type probe complementary to a mutant-type target base sequence and a fragment complementary to an adjacent base sequence adjacent to a 5′-terminal side of the target base sequence;
a step of producing a wild-type polynucleotide including the wild-type target base sequence, by ligating the wild-type probe and the fragment hybridized to the wild-type polynucleotide and producing a wild-type polynucleotide including the wild-type target base sequence by ligating the mutant-type probe and the fragment hybridized to the mutant-type polynucleotide; and
a step of fractionating the wild-type polynucleotide and the mutant-type polynucleotide by electrophoresis to determine a proportion of the mutant-type polynucleotide to the wild-type polynucleotide, wherein
the wild-type probe has a base sequence complementary to the wild-type target base sequence at a 3′-terminal side,
the mutant-type probe has a base sequence complementary to the mutant-type target base sequence at a 3′-terminal side, and
the plurality of wild-type probes have mutually different molecular structures that result in different mobilities in the electrophoresis.

10. The nucleic acid quantification method according to claim 9, wherein
the wild-type probe has a mobility correction site that corrects a mobility in the electrophoresis, and
the mobility correction site is formed by polynucleotides.

11. The nucleic acid quantification method according to claim 9, wherein
the wild-type probe has a mobility correction site that corrects the mobility in the electrophoresis, and
the mobility correction site is formed by a non-polynucleotide polymer.

12. The nucleic acid quantification method according to claim 9, wherein
the wild-type probe and the mutant-type probe are labeled with respective fluorescent dyes.

13. The nucleic acid quantification method according to claim 9, wherein
the wild-type probe and the mutant-type probe are labeled with respective radioisotopes.

14. A reagent for quantifying nucleic acid that quantifies a proportion of a mutant-type base sequence to a wild-type base sequence contained in a sample, comprising:
a plurality of wild-type forward primers complementary to a wild-type target base sequence;
a mutant-type forward primer complementary to a mutant-type target base sequence; and
a reverse primer complementary to a complementary strand of the target base sequence, wherein
the wild-type forward primer has a base sequence complementary to the wild-type target base sequence at a 3′-terminal side,
the mutant-type forward primer has a base sequence complementary to the mutant target base sequence at a 3′-terminal side, and
the plurality of wild-type forward primers have mutually different molecular structures that result in different mobilities in the electrophoresis.

15. A reagent for quantifying nucleic acid that quantifies a proportion of a mutant-type base sequence to a wild-type base sequence contained in a sample, comprising:
a plurality of wild-type probes complementary to a wild-type target base sequence;
a plurality of mutant-type probes complementary to a mutant-type target base sequence; and
a fragment complementary to an adjacent base sequence adjacent to a 5′-terminal side of the target base sequence, wherein
the wild-type probe has a base sequence complementary to the wild-type target base sequence at a 3′-terminal side,
the mutant-type probe has a base sequence complementary to the mutant-type target base sequence at a 3′-terminal side, and
the plurality of wild-type probes have mutually different molecular structures that result in different mobilities in the electrophoresis.
